(19) United States
(12) Patent Application Publication
(10) Pub. No.: US 2023/0028028 A1
(43) Pub. Date: Jan. 26, 2023

(54) Affinity Membranes, Compounds, Compositions and Processes for Their Preparation and Use

(71) Applicants: Fujifilm Manufacturing Europe B.V., Tilburg (NL); Fujifilm Corporation, Tokyo (JP)

(72) Inventors: Berry Bogels, Tilburg (NL); Yujiro Itami, Tilburg (NL)

(21) Appl. No.: 17/777,338

(22) PCT Filed: Nov. 24, 2020
(86) PCT No.: PCT/EP2020/083121

§ 371 Date: May 17, 2022

(30) Foreign Application Priority Data
Dec. 4, 2019 (GB) 1917710.4

(51) Int. Cl. B01D 67/00; C07K 1/22; C12N 15/10; B01D 69/10; B01D 69/02; B01D 15/34; B01D 15/36; B01D 61/14

(52) U.S. Cl. CPC B01D 67/0006; C07K 1/22; C12N 15/101; B01D 69/10; B01D 69/02; B01D 15/34; B01D 15/361; B01D 61/147; B01D 2323/30; B01D 2323/345; B01D 2325/12; B01D 2325/02

(57) ABSTRACT

A porous membrane obtainable by a process comprising curing a composition comprising: (i) cross-linking agent(s) comprising at least one ligand group; (ii) inert solvent(s); (iii) polymerization initiator(s); and (vi) optionally monomer(s) other than component (i) which are reactive with component (i); wherein the composition satisfies the following equation: Z=wt(i)/(wt(i)+wt(iii)+wt(iv)) wherein: Z has a value of at least 0.6; wt(i) is the number of grammes of component (i) present in the composition; wt(iii) is the number of grammes of component (iii) present in the composition; and wt(iv) is the number of grammes of component (iv) present in the composition.

The present invention relates to porous membranes, to compounds and to their preparation and use, e.g. for purifying biomolecules and compositions comprising metal ions.

A number of techniques are known for the purification of biomolecules (e.g. proteins, amino acids, nucleic acids, anti-bodies and endotoxins). These techniques include the use of affinity membrane filtration (AMF). In AMF impure compositions comprising biomolecules are contacted with a stationary phase (an affinity membrane) and the desired biomolecules can be separated from the impurities based on the higher affinity of the desired biomolecules for the membrane.

AMF may also be used for the purification of mixtures comprising metals, e.g. by using membranes having a higher affinity for some metals compared to others. AMF may be used, for example, to remove heavy metals from waste streams, water or organic solvents.

The key to efficient AMF is the preparation of porous membranes having a high affinity for a target chemicals (affinity membranes). In general, two approaches have been employed to prepare affinity membranes. In the most common method, affinity membranes are prepared from polyethylene, polypropylene, nylon, polysulfone, and glass. However, these membranes are usually hydrophobic and relatively inert, and hence require difficult (chemical) modifications. In addition, some of the membranes have low numbers of ligand groups due to the harsh chemical post-treatments required to introduce the ligands. To overcome these drawbacks, a second approach has been employed wherein membranes are prepared that have pre-incorporated ligand groups. However, the problems with this type of membrane include high hydrophobicity and brittleness. Another drawback with both of the above methods is that the pore size of the membrane cannot be easily controlled and the membranes typically have low water flux, low porosity, low capacity for removing target materials and often suffer from excessive swelling in aqueous media.

There is a need for affinity membranes that do not swell excessively when in contact with water and have good porosity, high capacity for removing target materials, good waterflux and a very high pore surface area.

According to a first aspect of the present invention there is provided a porous membrane obtainable by a process comprising curing a composition comprising:
(i) cross-linking agent(s) comprising at least one ligand group;
(ii) inert solvent(s);
(iii) polymerization initiator(s); and
(iv) optionally monomer(s) other than component (i) which are reactive with component (i);
   wherein the composition satisfies the following equation:

Z=wt(i)/(wt(i)++wt(iv))

wherein:
Z has a value of at least 0.6;
wt(i) is the number of grammes of component (i) present in the composition;
wt(iii) is the number of grammes of component (iii) present in the composition; and
wt(iv) is the number of grammes of component (iv) present in the composition.

Preferably component (i) (and optionally component (iv) when present) is completely dissolved in component (ii) and the membrane is insoluble in component (ii).

In this specification (including its claims), the verb “comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The cross-linking agent(s) preferably comprise at least two polymerisable groups, e.g. at least two groups selected from epoxy, thiol (—SH), oxetane and especially ethylenically unsaturated groups. The polymerisable groups will typically be selected such they are reactive with each other and/or with at least one polymerisable group present in another, chemically different component (i) (when present). When component (iv) is present the polymerisable groups of the cross-linking agent are preferably reactive with component (iv).

Curing causes the cross-linking agent to cross-link, e.g. to form the membrane as a cross-linked, three dimensional polymer matrix.

The at least two polymerisable groups present in component (i) may all be chemically identical or they may be different.

Preferred polymerisable groups are ethylenically unsaturated groups, especially (meth)acrylic groups and/or vinyl groups (e.g. vinyl ether groups, aromatic vinyl compounds, N-vinyl compounds and allyl groups).

Examples of suitable (meth)acrylic groups include acrylate (H₂C═CHCO—) groups, acrylamide (H₂C═CHCONH—) groups, methacrylate (H₂C═C(CH₃)CO—) groups and methacrylamide (H₂C═C(CH₃)CONH—) groups. Acrylic groups are preferred over methacrylic groups because acrylic groups are more reactive.

Preferred ethylenically unsaturated groups are free from ester groups because this can improve the stability and the pH tolerance of the resultant membrane. Ethylenically unsaturated groups which are free from ester groups include (meth)acrylamide groups and vinyl ether groups ((meth)acrylamide groups are especially preferred).

As preferred examples of polymerisable groups there may be mentioned groups of the following formulae:

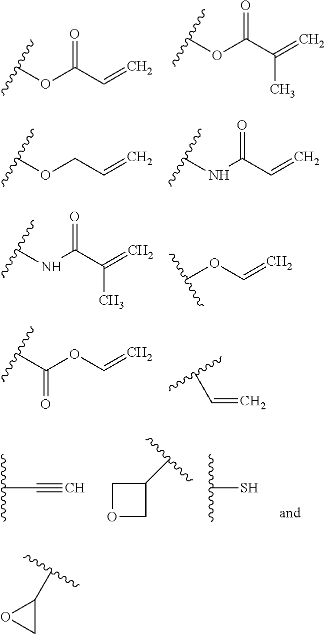

In one embodiment component (i) is free from sulphonic acid groups and/or comprises a heterocyclic ring.

The cross-linking agent(s) comprising at least one ligand group preferably is or comprises a compound of the Formula (1) or Formula (2) (such compounds and their use for preparing membranes (especially affinity membranes) forming further features of the present invention):

(L)_{q}-(A)ₘ-(R)ₙ Formula (1)

(R)ₙ₋₁-(A)ₘ-(L)_{q}-(A)ₘ-(R)ₙ₋₁ Formula (2)

wherein:

each L independently is a ligand group;

each A independently is an organic linking group;

each R independently is polymerisable group;
q has a value of at least 1;

each m independently has a value of 0 or 1; and

each n independently has a value of at least 2.

In Formula (2), preferably A and/or L comprises a phosphato group, a sulphonic acid group and/or a carboxy group, more preferably.

Preferably the organic linking group(s) represented by A each independently comprise from 2 to 12 carbon atoms (e.g. from two to twelve —CH₂— groups) and optionally one or more (e.g. 1 to 10) hetero atoms (e.g. nitrogen (e.g. —NH—) and/or oxygen (e.g. —O—) and/or sulfur (e.g. —S—)). Thus the organic linking group A (when present) is preferably a backbone to which the ligand group(s) L and the polymerisable groups R are attached. When each and every m has a value of zero the organic linking group A is absent from the compound of Formula (1) or (2) and the polymerisable groups are attached directly to the ligand group(s) L, e.g. through a single covalent bond.

Preferably q has a value of 1, 2, 3 or 4, especially 1, 2 or 3.

Preferably each n independently has a value of 2 or 3, especially 2.

Preferably the polymerisable groups represented by R are an ethylenically unsaturated groups.

In Formula (1) and Formula (2) each R independently preferably comprises a vinyl group, especially a (meth)acrylic group, a vinyl ether (H₂C═CHO—) group, an allyl ether (H₂C═CHCH₂O—) group or a styryl (H₂CH═CHC₆H₄—) group. Preferred (meth)acrylic groups include acrylate (H₂C═CHCO—) groups and methacrylate (H₂C═C(CH₃)CO—) groups and especially acrylamide (H₂C═CHCONH—) groups and methacrylamide (H₂C═C(CH₃)CONH—) groups. The compounds of Formula (1) and Formula (2) may be obtained by attaching ligand group(s) L and polymerisable groups R to an organic linking group, e.g. by a process comprising amide formation. For example, ligand groups carrying an acid chloride group and polymerisable groups carrying an acid chloride group may be reacted with an organic linking group having amine substituents, typically under mildly alkaline conditions, to form an amide bond between the ligand groups and the organic linking group and between the polymerisable groups and the organic linking group.

The ligand group can bind to target biomolecules or metal atoms covalently or, more typically, non-covalently. The ligand group can form a coordination complex with metal ions, in particular alkali metals, alkaline earth metals, lanthanide, actinide, transition metals, post-transition metals and metalloids. The bonding typically involves donation of 1 or more of the ligand group's free electron pairs to the metal ion. Preferably the ligand group is non-ionic or non-charged and has a binding constant for a metal with a valency of 1 or more or for a biomolecule (especially a biomolecule comprising amino acids) of at least 10⁴ M⁻¹ (especially 10⁴ M⁻¹ to 10³⁰ M⁻¹). One may use ligand groups having a variety of different degrees of bulkiness (size), a variety of different coordination atoms and a variety of different electrons which may be donated to a metal (denticity and hapticity). Preferably the ligand group can bind biomolecules by forming a ligand-biomolecule complex, e.g. a ligand-protein or ligand-DNA complex. Binding of the ligand group with biomolecules typically occurs by a non-covalent interaction, for example hydrogen-bonding, a pi-pi interaction, van der Waals forces, a hydrophobic effect, a host-guest interaction, halogen bonding, a dipole-dipole interaction, a dipole-induced dipole interaction or by two or more of the foregoing binding mechanisms. The affinity of the ligand group for particular biomolecule(s) or metal(s) enables the membrane to be used in AMF. For example, one may use a ligand which has a high affinity for (strepta)avidin-labeled biomolecules and a lower affinity for biomolecules which are not labeled with (strepta)avidin and in this case the membrane may be used to separate (strepta)avidin-labeled biomolecules from biomolecules which are not labeled with (strepta)avidin.

In one embodiment the ligand group is neutral and non-ionic in nature. Herein acidic and basic groups in the neutral form are considered to be non-ionic in nature.

Preferred heterocyclic rings are cyclic ethers and 5- or 6-membered rings comprising 1, 2 or 3 atoms selected from oxygen, sulphur and nitrogen.

Examples of heterocyclic rings include pyridyl (e.g. 2,2′-bipyridyl and picolinic acid groups); crown ethers; cryptands; cyclams; cyclens; siderophores (e.g. heterocyclic groups comprising catechol and/or hydroxamate groups); bipyrimidines; terpyridines; sarcophagines (e.g. calix[4]arene, carcerand, cavitand and phytochelatin groups); porphine clathrochelates; corroles; phtalocyanines; corrins; salens; and cyclic biomolecule-binding groups (e.g. peptide, (strepta)avidin, biotin, curcurbituril and cyclodextrin groups).

Optionally the heterocyclic ring has one or more metal-chelating substituents, for example, hydroxy (e.g. vicinal diol); thiol (e.g. vicinal dithiol); 1,3-diketone groups; amidoxime groups; amine (e.g. vicinal diamine); carboxylic acid groups; phosphoric acid groups; and/or sulphonic acid groups.

Crown ethers have a particularly high affinity for alkali metals, pyridyl groups have a particularly high affinity for transition metals and carboxylic acid groups have a high affinity for all metals.

Preferably heterocyclic rings include pyridyl groups, pyranyl groups, cyclic ether groups (e.g. crown ether groups, pyranyl groups and tetrahydropyranyl groups) and biotin groups, e.g. groups having at least one of the following formulae (wherein n has a value of from 1 to 3):

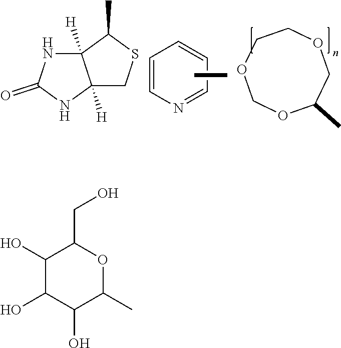

In a preferred embodiment component (i) comprises a backbone, at least two polymerisable groups and at least one (more preferably at least two, (e.g. two, three or four) ligand group(s) attached to the backbone. The backbone preferably comprises an alkylene group, especially an alkylene group comprising from 2 to 10, especially 2 to 8 carbon atoms.

In one embodiment L comprises a heterocyclic, carboxy and/or phosphato group. For example, the compounds of Formula (2) comprise at least one group, more preferably at least two groups, selected from carboxy groups and phosphato groups and salts thereof.

Preferred examples of component (i) include the compounds (M1) to (M36), (M38), (M40) and (M42) to (M48) below:

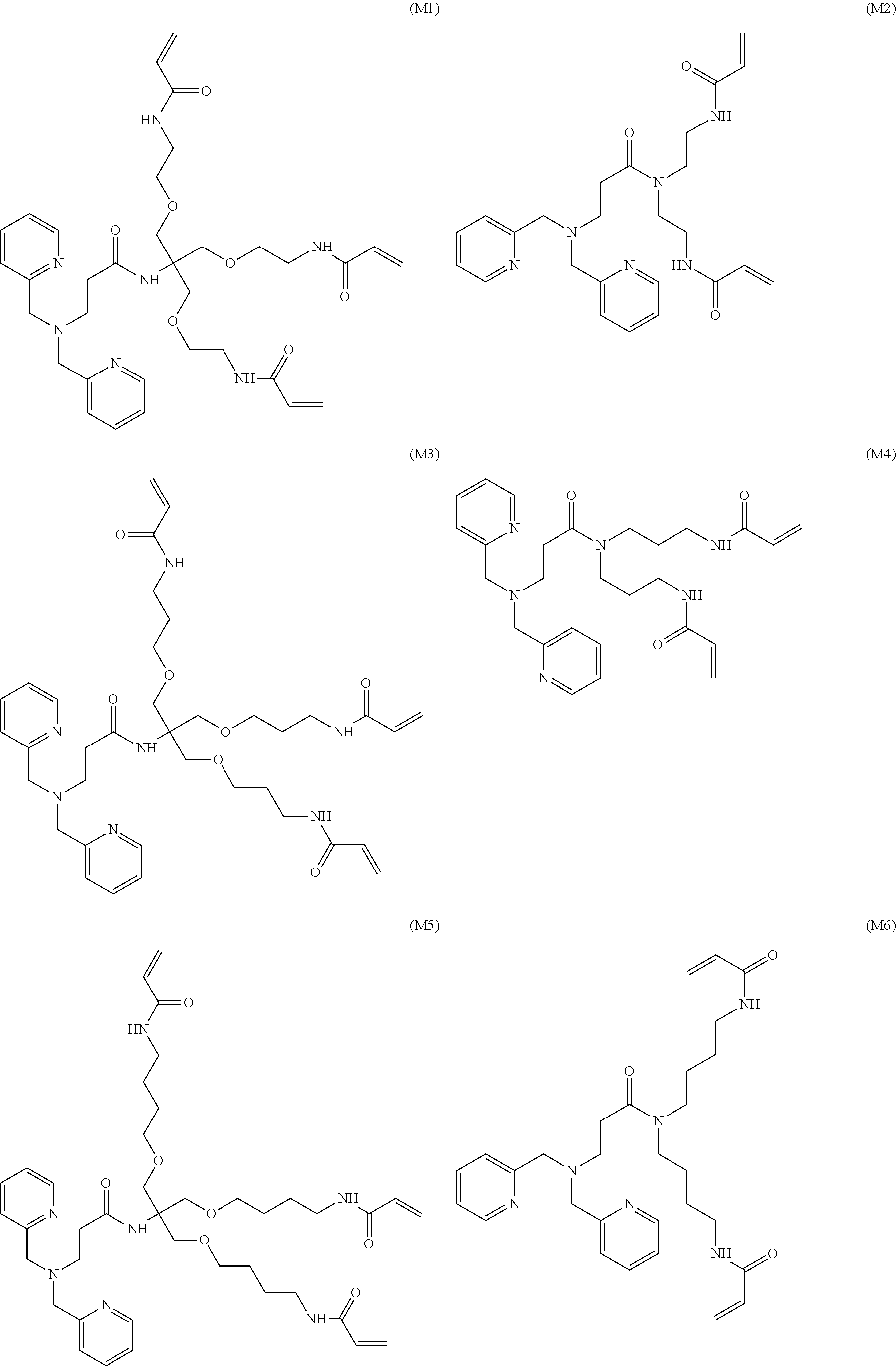

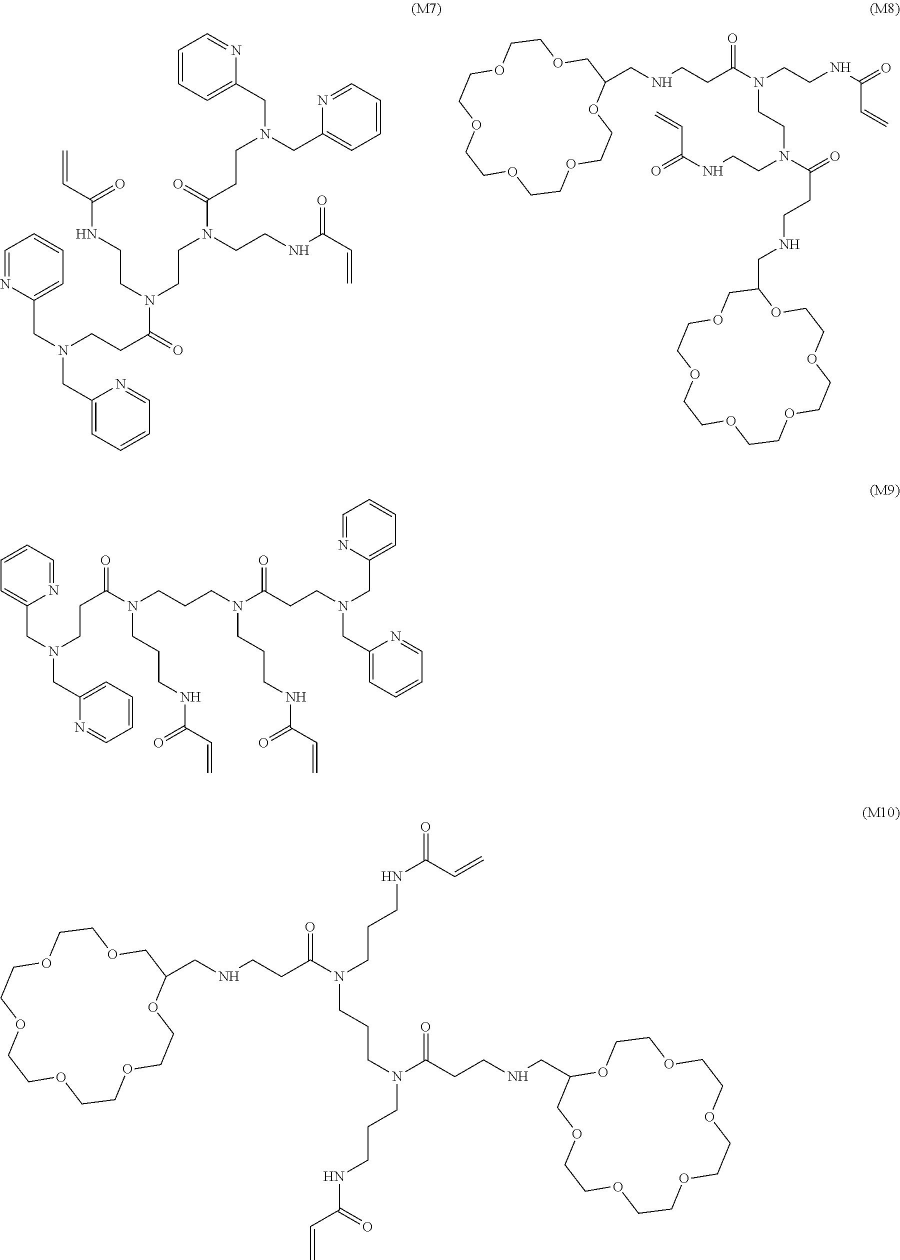 [SMILES: C=CC(=O)NCCN(CCN(CCNC(=O)C=C)C(=O)CCN(Cc1ccccn1)Cc1ccccn1)C(=O)CCN(Cc1ccccn1)Cc1ccccn1 ; C=CC(=O)NCCN(CCN(CCNC(=O)C=C)C(=O)CCNCC1COCCOCCOCCOCCOCCO1)C(=O)CCNCC1COCCOCCOCCOCCOCCO1 ; C=CC(=O)NCCCN(CCCN(CCCNC(=O)C=C)C(=O)CCN(Cc1ccccn1)Cc1ccccn1)C(=O)CCN(Cc1ccccn1)Cc1ccccn1 ; C=CC(=O)NCCCN(CCCN(CCCNC(=O)C=C)C(=O)CCNCC1COCCOCCOCCOCCOCCO1)C(=O)CCNCC1COCCOCCOCCOCCOCCO1]

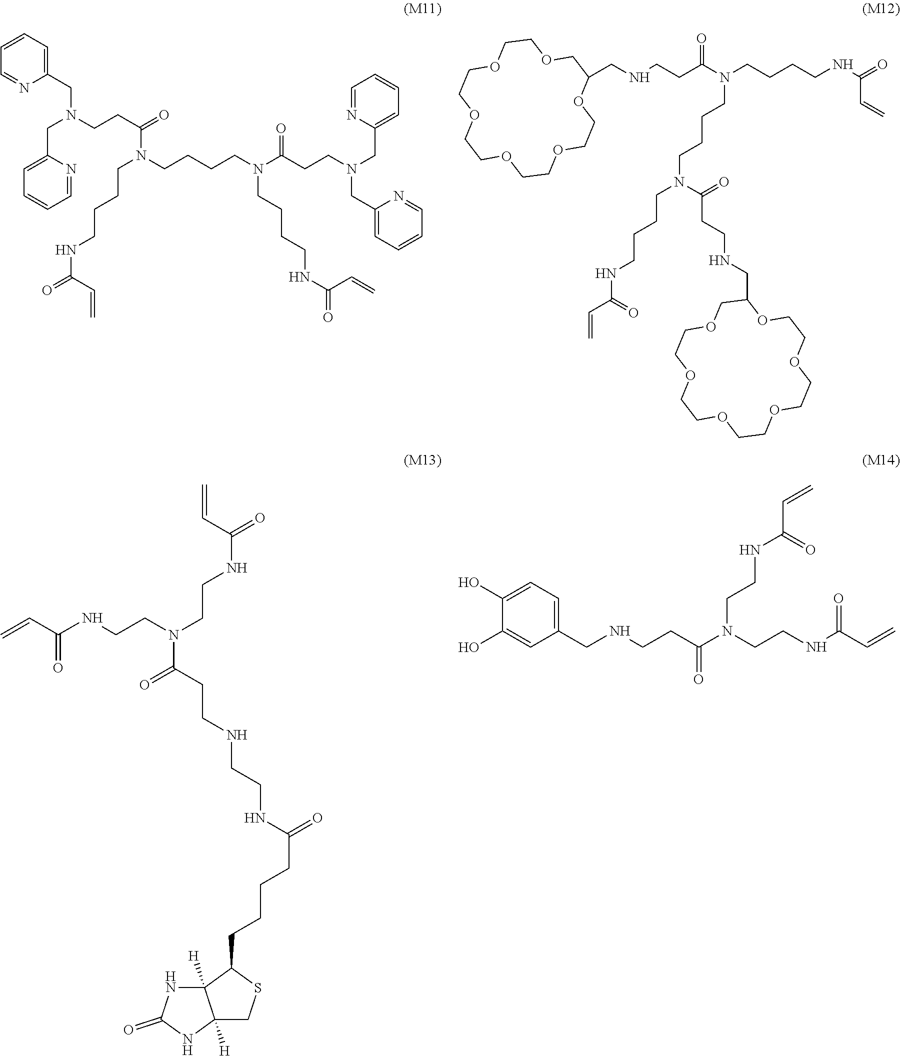 [SMILES: C=CC(=O)NCCCCN(CCCCN(CCCCNC(=O)C=C)C(=O)CCN(Cc1ccccn1)Cc1ccccn1)C(=O)CCN(Cc1ccccn1)Cc1ccccn1 ; C=CC(=O)NCCCCN(CCCCN(CCCCNC(=O)C=C)C(=O)CCNCC1COCCOCCOCCOCCOCCO1)C(=O)CCNCC1COCCOCCOCCOCCOCCO1 ; C=CC(=O)NCCN(CCNC(=O)C=C)C(=O)CCNCCNC(=O)CCCC[C@H]1SC[C@H]2NC(=O)N[C@H]21 ; C=CC(=O)NCCN(CCNC(=O)C=C)C(=O)CCNCc1ccc(O)c(O)c1]

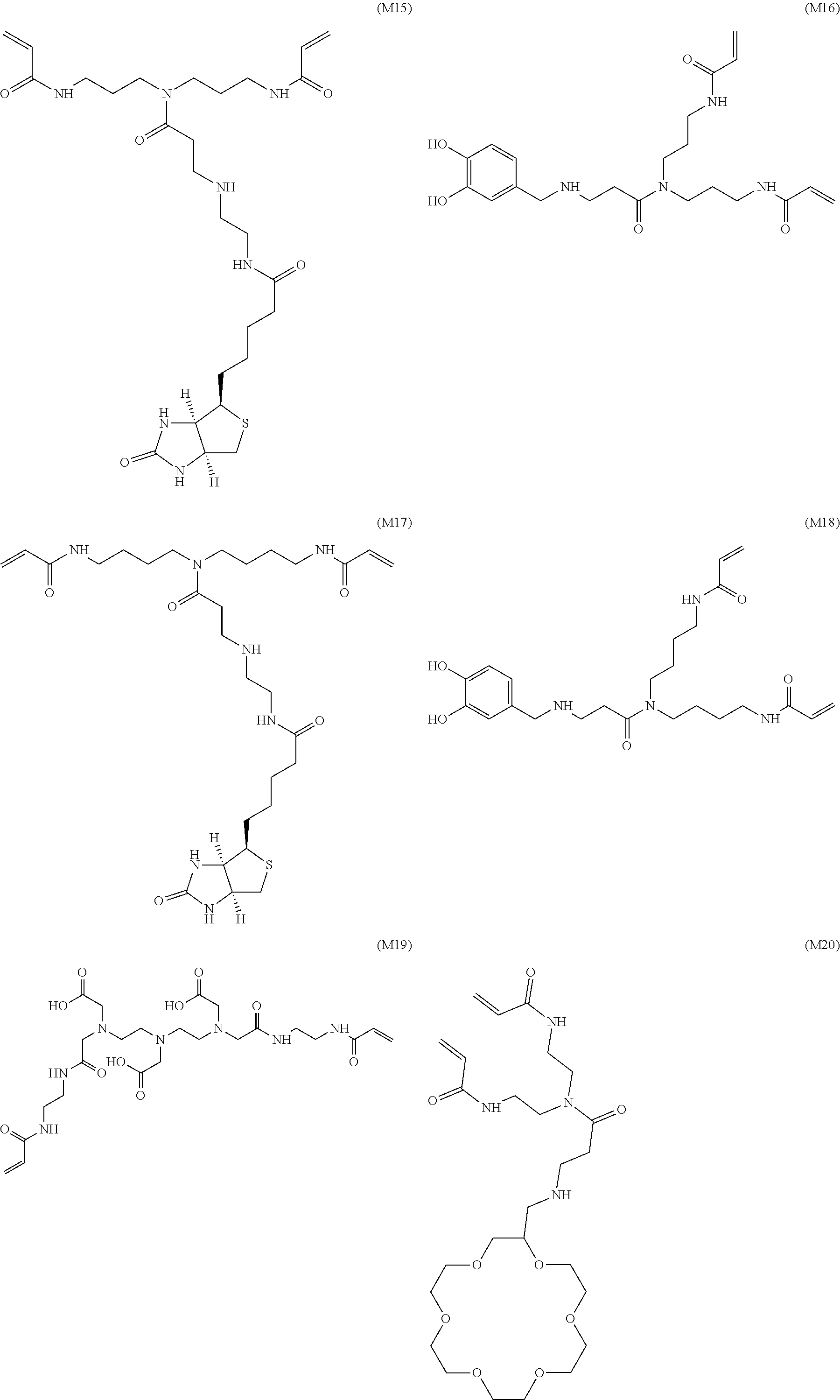 [SMILES: C=CC(=O)NCCCN(CCCNC(=O)C=C)C(=O)CCNCCNC(=O)CCCC[C@H]1SC[C@H]2NC(=O)N[C@H]21 ; C=CC(=O)NCCCN(CCCNC(=O)C=C)C(=O)CCNCc1ccc(O)c(O)c1 ; C=CC(=O)NCCCCN(CCCCNC(=O)C=C)C(=O)CCNCCNC(=O)CCCC[C@H]1SC[C@H]2NC(=O)N[C@H]21 ; C=CC(=O)NCCCCN(CCCCNC(=O)C=C)C(=O)CCNCc1ccc(O)c(O)c1 ; C=CC(=O)NCCNC(=O)CN(CCN(CCN(CC(=O)O)CC(=O)NCCNC(=O)C=C)CC(=O)O)CC(=O)O ; C=CC(=O)NCCN(CCNC(=O)C=C)C(=O)CCNCC1COCCOCCOCCOCCOCCO1]

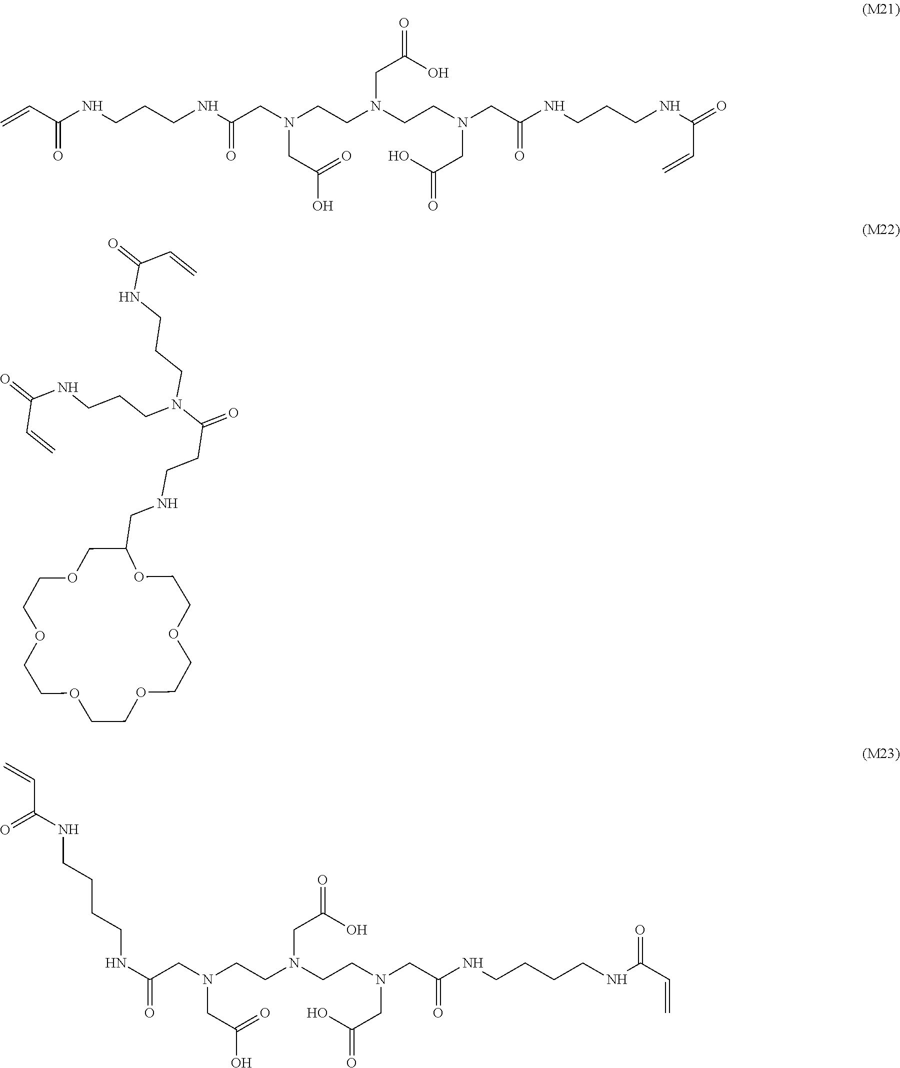 [SMILES: C=CC(=O)NCCCNC(=O)CN(CCN(CCN(CC(=O)O)CC(=O)NCCCNC(=O)C=C)CC(=O)O)CC(=O)O ; C=CC(=O)NCCCN(CCCNC(=O)C=C)C(=O)CCNCC1COCCOCCOCCOCCOCCO1 ; C=CC(=O)NCCCCNC(=O)CN(CCN(CCN(CC(=O)O)CC(=O)NCCCCNC(=O)C=C)CC(=O)O)CC(=O)O]

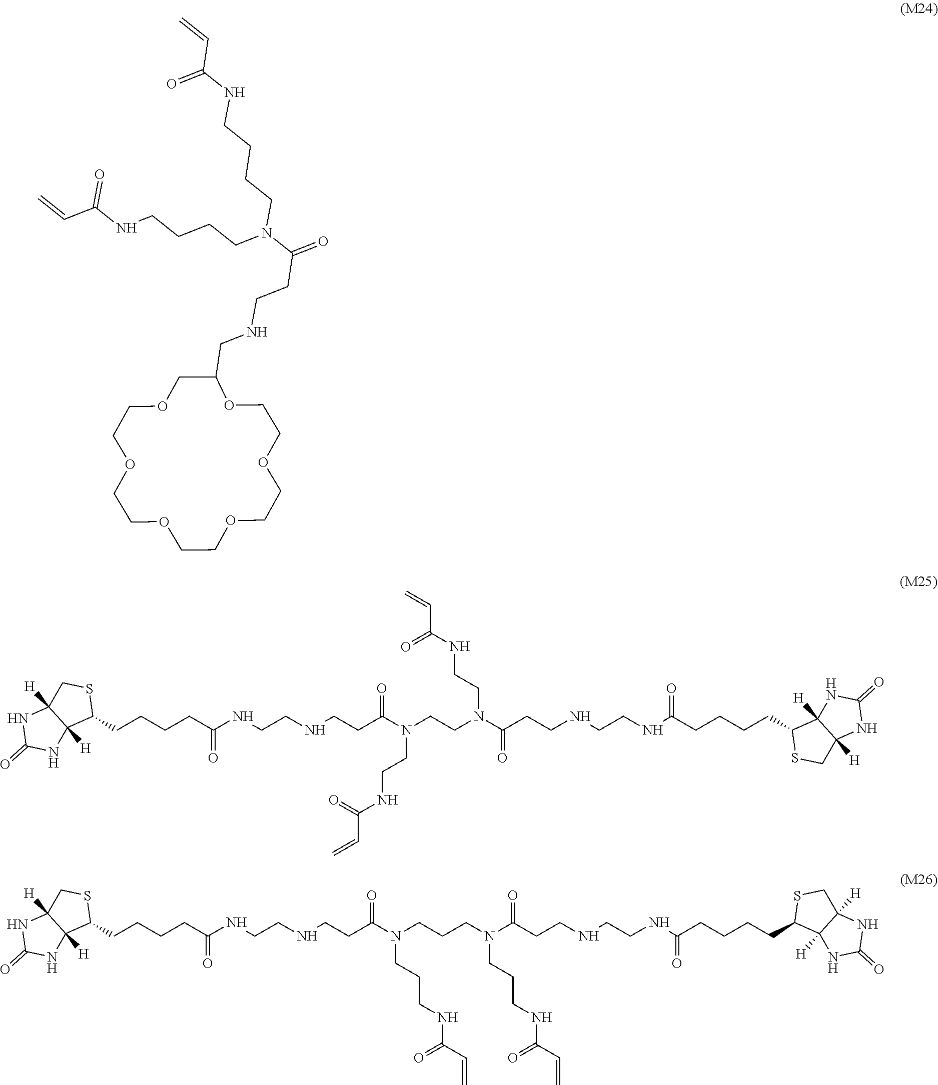 [SMILES: C=CC(=O)NCCCCN(CCCCNC(=O)C=C)C(=O)CCNCC1COCCOCCOCCOCCOCCO1 ; C=CC(=O)NCCN(CCN(CCNC(=O)C=C)C(=O)CCNCCNC(=O)CCCC[C@H]1SC[C@H]2NC(=O)N[C@H]21)C(=O)CCNCCNC(=O)CCCC[C@H]1SC[C@H]2NC(=O)N[C@H]21 ; C=CC(=O)NCCCN(CCCN(CCCNC(=O)C=C)C(=O)CCNCCNC(=O)CCCC[C@H]1SC[C@H]2NC(=O)N[C@H]21)C(=O)CCNCCNC(=O)CCCC[C@H]1SC[C@H]2NC(=O)N[C@H]21]

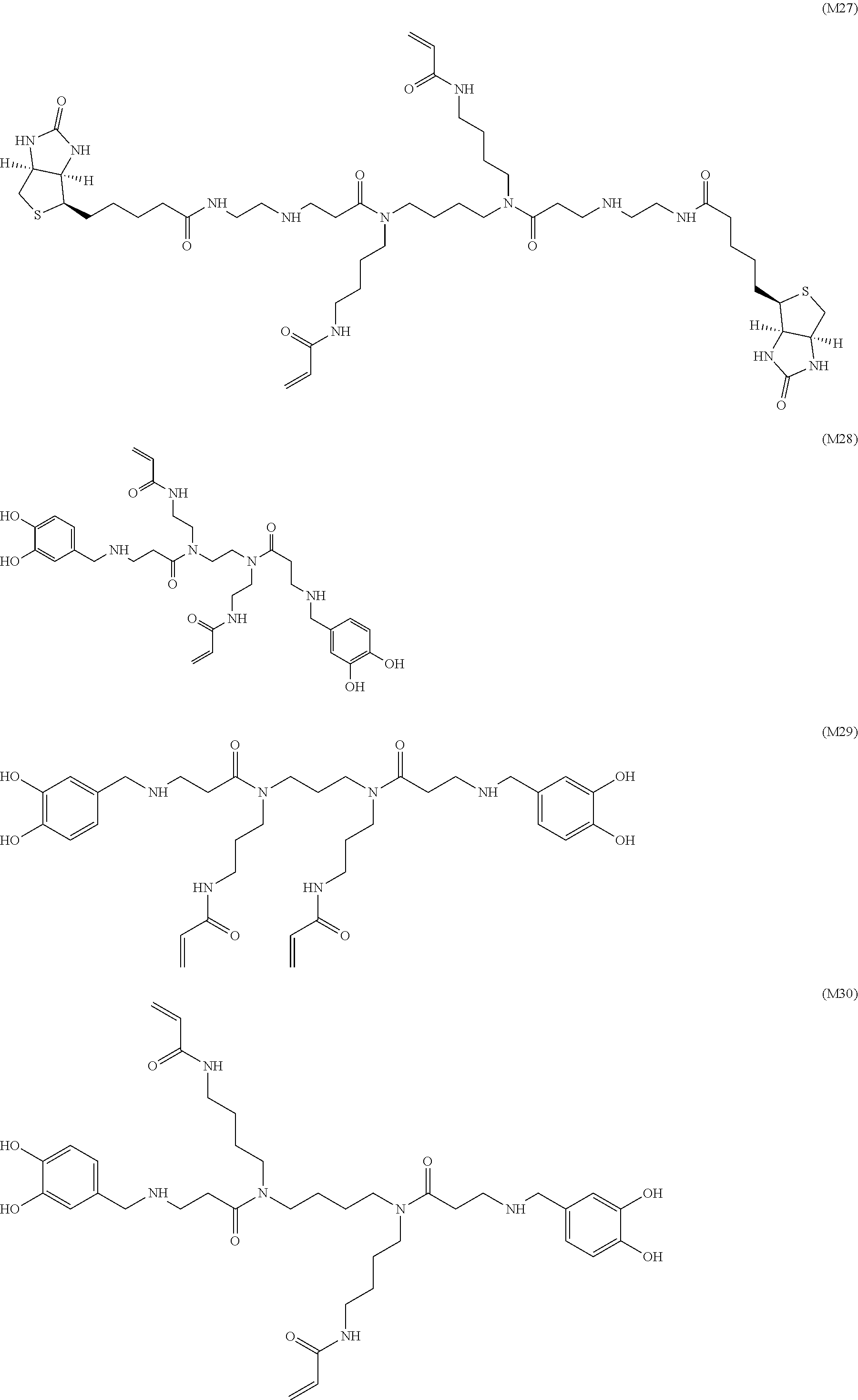 [SMILES: C=CC(=O)NCCCCN(CCCCN(CCCCNC(=O)C=C)C(=O)CCNCCNC(=O)CCCC[C@H]1SC[C@H]2NC(=O)N[C@H]21)C(=O)CCNCCNC(=O)CCCC[C@H]1SC[C@H]2NC(=O)N[C@H]21 ; C=CC(=O)NCCN(CCN(CCNC(=O)C=C)C(=O)CCNCc1ccc(O)c(O)c1)C(=O)CCNCc1ccc(O)c(O)c1 ; C=CC(=O)NCCCN(CCCN(CCCNC(=O)C=C)C(=O)CCNCc1ccc(O)c(O)c1)C(=O)CCNCc1ccc(O)c(O)c1 ; C=CC(=O)NCCCCN(CCCCN(CCCCNC(=O)C=C)C(=O)CCNCc1ccc(O)c(O)c1)C(=O)CCNCc1ccc(O)c(O)c1]

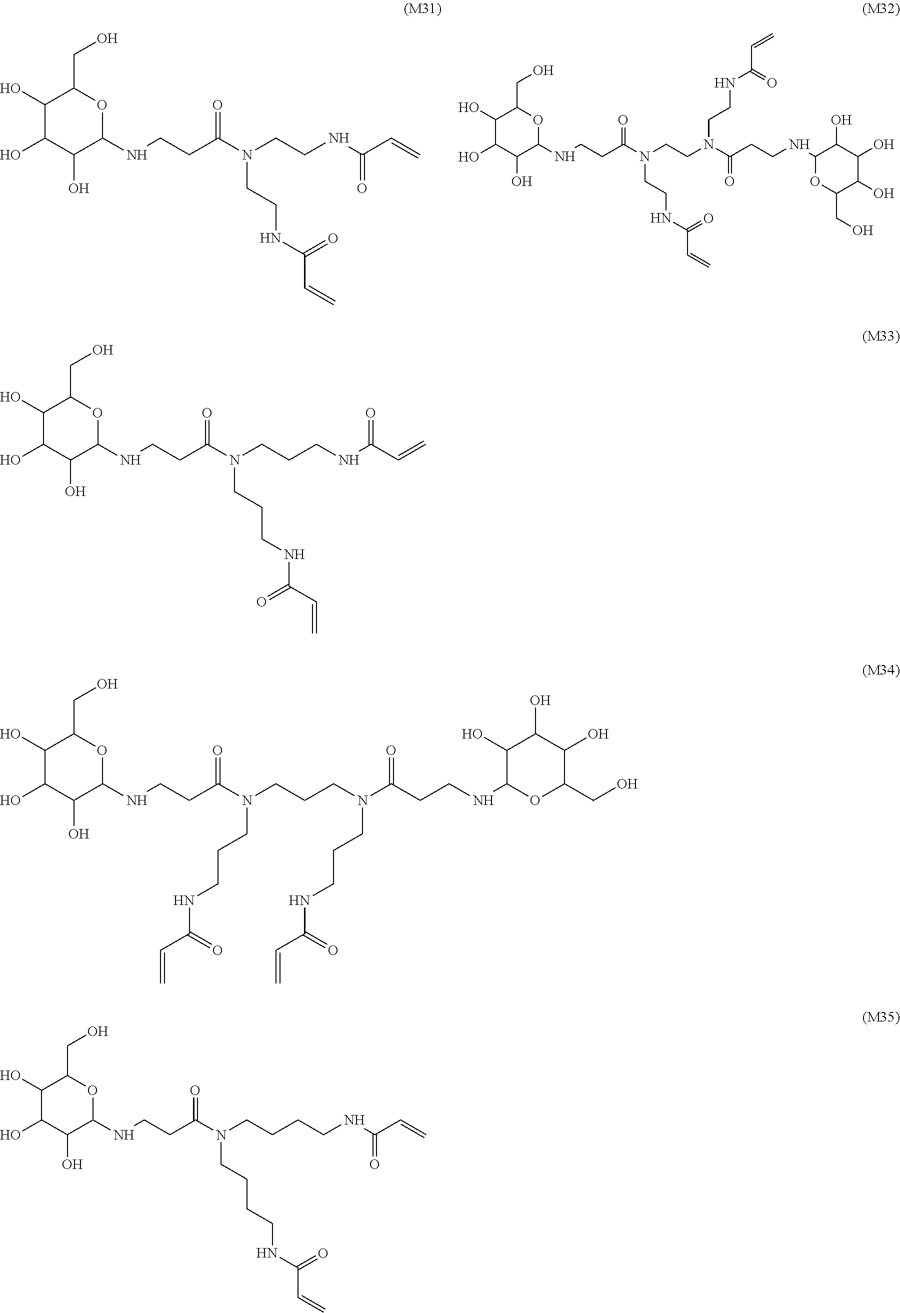 [SMILES: C=CC(=O)NCCN(CCNC(=O)C=C)C(=O)CCNC1OC(CO)C(O)C(O)C1O ; C=CC(=O)NCCN(CCN(CCNC(=O)C=C)C(=O)CCNC1OC(CO)C(O)C(O)C1O)C(=O)CCNC1OC(CO)C(O)C(O)C1O ; C=CC(=O)NCCCN(CCCNC(=O)C=C)C(=O)CCNC1OC(CO)C(O)C(O)C1O ; C=CC(=O)NCCCN(CCCN(CCCNC(=O)C=C)C(=O)CCNC1OC(CO)C(O)C(O)C1O)C(=O)CCNC1OC(CO)C(O)C(O)C1O ; C=CC(=O)NCCCCN(CCCCNC(=O)C=C)C(=O)CCNC1OC(CO)C(O)C(O)C1O]

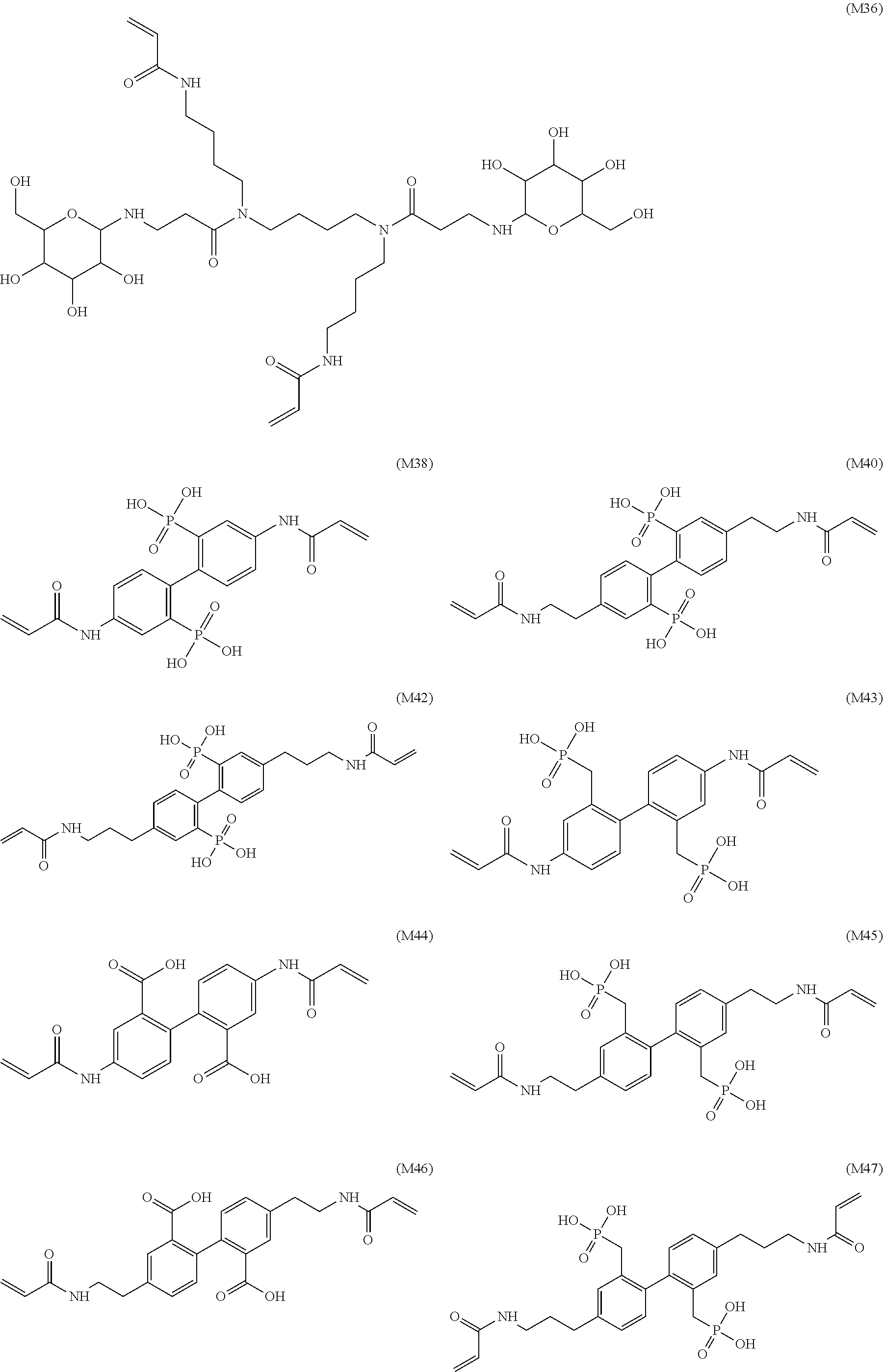

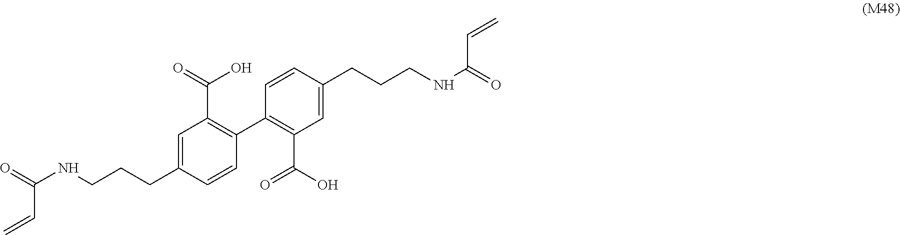 [SMILES: C=CC(=O)NCCCc1ccc(-c2ccc(CCCNC(=O)C=C)cc2C(=O)O)c(C(=O)O)c1]

In a preferred embodiment each L independently comprises a pyridyl group (especially two pyridyl groups), a crown ether group, a 3,4-dihydroxybenzyl group, a pyranyl group having two or three hydroxy substituents or a biotin group.

Examples of compounds of Formula (1) in which each L comprises a pyridyl group (in fact two pyridyl groups) include (M1) to (M6), (M7), (M9) and (M11) shown above.

Examples of compounds of Formula (1) in which each L comprises a crown ether group include (M8), (M10), (M12), (M20), (M22) and (M24) shown above.

Examples of compounds of Formula (1) in which each L comprises a 3,4-dihydroxybenzyl group include (M14), (M16), (M18) and (M28) to (M30) shown above.

Examples of compounds of Formula (1) in which each L comprises a pyranyl group having two or three hydroxy substituents include (M31) to (M36) shown above.

Examples of compounds of Formula (1) in which each L comprises a biotin group include (M13), (M15), (M17) and (M25) to (M27) shown above.

Examples of compounds of Formula (2) include (M19), (M21), (M23), (M38), (M40), (M42) to (M48) shown above.

The amount of component (i) present in the composition, relative to the total weight of the composition, is preferably 6 to 80 wt %, more preferably 6 to 60 wt %, and especially 20 to 40 wt %. When the composition contains less than 20 wt % of component (i), components other than component (ii) make up the balance to 100 wt %, e.g. components (iii) and/or (iv), as described in more detail below. Preferably component (i) is completely dissolved in the composition (e.g. in component (ii)).

Preferably component (i) has a molecular weight below 2,000 Da, more preferably 1,500 Da or below and especially 100 Da to 1,500 Da.

As mentioned above, the compounds of Formula (1) and (2) as defined above form, a further feature of the present invention. In this further feature of the present invention it is preferred that each L independently is as defined above. Still further, the compositions defined in the first aspect of the present invention which contain the compounds of Formula (1) and/or Formula (2), as defined above, form a further feature of the present invention.

In this specification “inert” means non-polymerisable. Thus component (ii) is incapable of polymerising with component (i).

Component (ii) preferably consists of water and one or more water-miscible organic solvents. The inert character of component (ii) assists the formation of pores in the membrane.

Preferably the wt % of water in the composition (and component (ii)) is lower than the wt % of the water-miscible organic solvent.

Preferably component (ii) is a non-solvent for the membrane (i.e. preferably the membrane is insoluble in component (ii)). Component (ii) performs the function of dissolving component (i) and preferably also components (iii) and (iv) (when present). Component (ii) can also help to ensure that the membrane precipitates from the composition as it is formed, e.g. by a phase separation process.

The amount of component (ii) present in the composition, relative to the total weight of the composition, is preferably 15.0 to 94.99 wt %, more preferably 38.0 to 94.99 wt % and especially 59.5 to 79.99 wt %.

Curing causes component (i) to cross-link, e.g. to form the membrane as a crosslinked, three dimensional polymer matrix.

Preferably component (ii) comprises water and a water-miscible organic solvent having a water-solubility of at least 5 wt %.

Examples of water-miscible organic solvents which may be used in component (ii) include alcohol-based solvents, ether-based solvents, amide-based solvents, ketone-based solvents, sulfoxide-based solvents, sulfone-based solvents, nitrile-based solvents and organic phosphorus-based solvents, of which inert, aprotic, polar solvents are preferred.

Examples of alcohol-based solvents which may be used as or in component (ii) (especially in combination with water) include methanol, ethanol, isopropanol, n-butanol, ethylene glycol, propylene glycol, diethylene glycol, dipropylene glycol and mixtures comprising two or more thereof. Isopropanol is particularly preferred.

In addition, preferred inert, organic solvents which may be used as or in component (ii) include dimethyl sulfoxide, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-m ethyltetrahydrofuran, ethylene glycol diacetate, cyclopentylmethylether, methylethylketone, ethyl acetate, γ-butyrolactone and mixtures comprising two or more thereof. Dimethyl sulfoxide, N-methyl pyrrolidone, dimethyl formamide, dimethyl imidazolidinone, sulfolane, acetone, cyclopentylmethylether, methylethylketone, acetonitrile, tetrahydrofuran, 2-methyltetrahydrofuran and mixtures comprising two or more thereof are preferable.

In a preferred embodiment component (ii) comprises water, one or more solvents selected from list (iia) and optionally one or more solvents selected from list (iib):

list (iia): iso-propanol, methanol, ethanol, acetone, tetramethyl urea, hexamethyl phosphoramide, hexamethyl phosphorotriamide, butanone, cyclohexanone, methylethylketone, tetrahydrofuran, tetrahydropyran, 2-methyltetrahydrofuran, cyclopentylmethylether, propionitrile, acetonitrile, 1,4-dioxane, 1,3-dioxolane, ethyl acetate, γ-butyrolactone and ethanolamine; and

list (iib): glycerol, ethylene glycol, dimethyl sulfoxide, sulpholane, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-m ethyltetrahydrofuran, ethylene glycol diacetate, cyclopentylmethylether, methylethylketone, ethyl acetate and γ-butyrolactone, and among these, dimethyl sulfoxide, N-methyl pyrrolidone, N,N-dimethyl formamide, dimethyl imidazolidinone, N-methyl morpholine, acetone, cyclopentylmethylether, methylethylketone, acetonitrile, tetrahydrofuran and 2-m ethyltetrahydrofuran.

In one embodiment the composition comprises water and one or more other solvents from list (iia) and/or list (iib).

In another embodiment component (ii) comprises 0 to 57 wt % of water, 0 to 72 wt % of solvent(s) selected from list (iia) and 0 to 72 wt % of solvent(s) selected from list (iib).

Thus a preferred composition comprises 6.0 to 80.0 wt % of component (i), 15.0 to 94.99 wt % of component (ii) and 0.01 to 5.0 wt % of component (iii), more preferably 6.0 to 60.0 wt % of component (i), 38.0 to 94.99 wt % of component (ii) and 0.01 to 2.0 wt % of component (iii), especially 20.0 to 40.0 wt % of component (i), 59.5 to 79.99 wt % of component (ii) and 0.01 to 5.0 wt % of component (iii). Preferably component (ii) comprises isopropanol and water, especially 40 to 73 wt % of isopropanol, and 27 to 60 wt % of water

Preferably the polymerisation initiator(s) comprise a thermal initiator and/or a photoinitiator.

Examples of suitable thermal initiators which may be included in the composition include 2,2′-azobis(2-methylpropionitrile) (AIBN), 4,4′-azobis(4-cyanovaleric acid), 2,2′-azobis(2,4-dimethyl valeronitrile), 2,2′-azobis(2-methylbutyronitrile), 1,1′-azobis(cyclohexane-1-carbonitrile), 2,2′-azobis(4-methoxy-2,4-dimethyl valeronitrile), dimethyl 2,2′-azobis(2-methylpropionate), 2,2′-azobis[N-(2-propenyl)-2-methylpropionamide, 1-[(1-cyano-1-methylethyl)azo]formamide, 2,2′-Azobis(N-butyl-2-methylpropionamide), 2,2′-Azobis(N-cyclohexyl-2-methylpropionamide), 2,2′-Azobis(2-methylpropionamidine) dihydrochloride, 2,2′-Azobis[2-(2-imidazolin-2-yl)propane]dihydrochloride, 2,2′-Azobis[2-(2-imidazolin-2-yl)propane]disulfate dihydrate, 2,2′-Azobis[N-(2-carboxyethyl)-2-methylpropionamidine] hydrate, 2,2′-Azobis{2-[1-(2-hydroxyethyl)-2-imidazolin-2-yl]propane} dihydrochloride, 2,2′-Azobis[2-(2-imidazolin-2-yl)propane], 2,2′-Azobis(1-imino-1-pyrrolidino-2-ethylpropane) dihydrochloride, 2,2′-Azobis{2-methyl-N-[1,1-bis(hydroxymethyl)-2-hydroxyethyl]propionamide} and 2,2′-Azobis[2-methyl-N-(2-hydroxyethyl)propionamide].

Examples of suitable photoinitiators which may be included in the composition include aromatic ketones, acylphosphine compounds, aromatic onium salt compounds, organic peroxides, thio compounds, hexaarylbiimidazole compounds, ketoxime ester compounds, borate compounds, azinium compounds, metallocene compounds, active ester compounds, compounds having a carbon halogen bond, and an alkyl amine compounds. Preferred examples of the aromatic ketones, the acylphosphine oxide compound, and the thio compound include compounds having a benzophenone skeleton or a thioxanthone skeleton described in “RADIATION CURING IN POLYMER SCIENCE AND TECHNOLOGY”, pp. 77-117 (1993). More preferred examples thereof include an alpha-thiobenzophenone compound described in JP1972-6416B (JP-S47-6416B), a benzoin ether compound described in JP1972-3981B (JP-S47-3981B), an alpha-substituted benzoin compound described in JP1972-22326B (JP-S47-22326B), a benzoin derivative described in JP1972-23664B (JP-S47-23664B), an aroylphosphonic acid ester described in JP1982-30704A (JP-557-30704A), dialkoxybenzophenone described in JP1985-26483B (JP-S60-26483B), benzoin ethers described in JP1985-26403B (JP-S60-26403B) and JP1987-81345A (JP-S62-81345A), alpha-amino benzophenones described in JP1989-34242B (JP-H01-34242B), U.S. Pat. No. 4,318,791A, and EP0284561A1, p-di(dimethylaminobenzoyl)benzene described in JP1990-211452A (JP-H02-211452A), a thio substituted aromatic ketone described in JP1986-194062A (JP-S61-194062A), an acylphosphine sulfide described in JP1990-9597B (JP-H02-9597B), an acylphosphine described in JP1990-9596B (JP-H02-9596B), thioxanthones described in JP1988-61950B (JP-S63-61950B), and coumarins described in JP1984-42864B (JP-S59-42864B). In addition, the photoinitiators described in JP2008-105379A and JP2009-114290A are also preferable. In addition, photoinitiators described in pp. 65 to 148 of “Ultraviolet Curing System” written by Kato Kiyomi (published by Research Center Co., Ltd., 1989) may be used.

The polymerisation initiator is preferably water-soluble. The polymerisation initiator (iii) preferably has a water-solubility of at least 1 wt %, more preferably at least 3 wt %, when measured at 25° C.

The composition preferably comprises 0.01 to 5 wt %, more preferably 0.01 to 2.0 wt %, and most preferably 0.01 to 0.5 wt % of the component (iii), based on the total weight of the composition.

Optionally the composition further comprises (iv) monomer(s) other than component (i) which is/are reactive with component (i), for example monomer(s) which comprises one (and only one) polymerisable group (e.g. an ethylenically unsaturated group) and optionally one or more ligand groups. Preferred polymerisable groups are ethylenically unsaturated groups and especially (meth)acrylic groups, as described above in relation to component

Preferably the number of moles of component (i) exceeds the number of moles of component (iv), when present.

The composition preferably comprises 0 to 20 wt % of component (iv), more preferably 0 to 15 wt %, especially 0 to 10 wt % of component (iv), based on the total weight of the composition.

In a preferred embodiment, Z has a value of at least 0.7, more preferably at least 0.8, especially at least 0.87, more especially at least 0.9, even more especially at least 0.95 and particularly at least 0.98. Preferably Z has a value of 0.999 or less. In a most preferred embodiment the composition is free from component (iv) (i.e. the composition is free from monomers other than component (i) which are reactive with component (i)).

Preferably component (iv), when present, is soluble in component (ii).

Optionally the composition includes one or more further components, e.g. a surfactant, a polymer dispersant, a polymerization reaction controlling agent, a thickening agent, an anti-crater agent, or the like, in addition to the above-described components.

The composition may be cured by any suitable process, including thermal curing, photocuring and combinations of the foregoing. However the composition is preferably cured by photocuring, e.g. by irradiating the composition and thereby causing component (i) and any other polymerisable components present in the composition to polymerise. Component (ii) is inert and does not polymerise, instead leaving pores in the resultant membrane.

Preferably the curing comprises photo-polymerization induced phase separation (“PIPS”) of the membrane from the composition.

PIPS, combined with selecting a suitable amount of component (i), component (ii), component (iii) and optionally component (iv), allows one to obtain particularly good membranes having good waterflux, porosity and low swelling in water. Using too much of component (i) and too little of component (ii) can result in a dense membrane with low waterflux, low porosity, and low swelling. Using too little of component (i) and too much of component (ii) can result in a dense membrane material having low waterflux, but high porosity and high swelling. The latter appears dense, however the pore structure tends to collapse and therefore forms a more dense material.

Thus in a preferred embodiment the composition comprises 6.0 to 60.0 wt % of component (i), 15.0 to 94.99 wt % of component (ii), 0.01 to 5.0 wt % of component (iii) and 0.0 to 20.0 wt % of component (iv), provided that Z (as hereinbefore defined) has a value of at least 0.6, and the curing comprises photo-polymerization induced phase separation of the membrane from the composition

According to a second aspect of the present invention there is provided a process for preparing a membrane according to the first aspect of the present invention comprising curing the composition defined in the first aspect of the present invention.

Preferably the membrane according to the first aspect of the present invention is obtained by a process comprising the steps of:
(a) mixing components (i), (ii), (iii) and optionally (iv) to form a composition comprising components (i), (ii), (iii) and optionally (iv) in which Z (as hereinbefore defined) has a value of at least 0.6;
(b) curing (e.g. irradiating) the composition arising from step (a) and thereby polymerising component (i) (and component (iv) when present) to form a membrane; and
(c) optionally washing the membrane arising from step (b).

The preferred compositions used in the process of the second aspect of the present invention are as described in relation to the first aspect of the present invention.

Optionally component (i) is free from ionic groups during step (a). In this embodiment, the process preferably further comprises the step of providing the ligand derived from component (i) with an ionic charge after performing step (i).

Preferably the process used to prepare the membranes of the present invention comprise polymerisation-induced phase separation, more preferably photo-polymerization induced phase separation, e.g. of the membrane from the composition. In this process, preferably the polymer is formed due to a photo-polymerization reaction.

Optionally step (b) may be performed by one or more further irradiation and/or heating steps in order to fully cure the membrane.

Including component (ii) in the composition has the advantage of helping the polymerisation in step (b) proceed uniformly and smoothly.

In a preferred embodiment component (ii) acts as a solvent for component (i) and assists the formation of the pores in the resultant membrane. Preferably component (ii) acts as a non-solvent for the resultant membrane.

The process according to the second aspect of the present invention provides substantially uniform membranes, often with a substantially uniform bicontinuous structure. In some embodiments the curing causes component (i) (and component (iv) when present) to form substantially uniform polymer particles which then merge to form the membranes of the present invention. The polymer particles, or agglomerates thereof, preferably have an average diameter in the range of 0.1 nm to 5,000 nm. Preferably the polymer particles have an average particle or agglomerate size of 1 nm to 2,000 nm, most preferably, 10 nm to 1,000 nm. The average particle or agglomerate size may be determined by cross-sectional analysis using Scanning Electron Microscopy (SEM).

Optionally the membrane of the present invention further comprises a support, especially a porous support. Inclusion of a support can provide the membrane with increased mechanical strength. If desired the composition may be applied to the support between steps (a) and (b) of the process for preparing the membranes according to the second aspect of the present invention. In this way the porous support may be impregnated with the composition and the composition may then be polymerised on and/or within the support.

Examples of suitable supports include synthetic woven fabrics and synthetic non-woven fabrics, sponge-like films, and films having fine through holes. The material for forming the optional porous support can be a porous membrane based on, for example, polyolefin (polyethylene, polypropylene, or the like), polyacrylonitrile, polyvinyl chloride, polyester, polyamide, or copolymers thereof, or, for example, polysulfone, polyether sulfone, polyphenylene sulfone, polyphenylene sulfide, polyimide, polyetheramide, polyamide, polyamideimide, polyacrylonitrile, polycarbonate, polyacrylate, cellulose acetate, cellulose, polypropylene, poly(4-methyl-1-pentene), polyvinylidene fluoride, polytetrafluoroethylene, polyhexafluoropropylene, polychlorotrifluoroethylene, or copolymers thereof. Among these, in the present invention, polyolefin, polyamide and cellulose are preferable.

As the commercially available porous support there may be used products from Japan Vilene Company, Ltd., Freudenberg Filtration Technologies, Sefar AG, Cerex Avanced Fabrics or Asahi-Kasei.

When the membrane comprises a support and the curing comprises photocuring then preferably the support does not shield the wavelength of light used to cure the composition.

The support is preferably a low leaching support, with organic or inorganic leachables below 1.0 ppb or not continuous leaching but to be lowered up to 1.0 ppb by extracting with water or organic solvent and/or hot water or organic solvent.

The membrane according to the present invention may optionally include more than one supports and the more than one support may be identical to each other or different.

Preferably the membrane comprises 60.0 to 99.99 wt % of component (i), 0.01 to 5.0 wt % of component (iii), and 0 to 35.0 wt % of component (iv). When the membrane comprises a support the aforementioned % relate to the part of the membrane other than the support.

The membrane preferably has a mean flow pore size of 5 to 5,000 nm, more preferably 100 to 2,000 nm. The mean flow pore size of the membrane according to the present invention may be measured using a porometer, e.g. a Porolux™ porometer. For example, one may fully wet the membrane to be tested with a wetting fluid (e.g. Porefil™ wetting Fluid, an inert, non-toxic, fluorocarbon wetting fluid with zero contact angle), place the wetted membrane in the sample holder of the porometer and apply a pressure of up to 35 mbar. The porometer can then provide the bubble point, maximum pore size, mean flow pore size, minimum pore size, average pore size distribution (of uniform materials) and air permeability of the membrane under test.

When the membrane does not comprise a support, the membrane preferably has a porosity of 15 to 99%, preferably 20 to 99% and especially 20 to 85%.

When the membrane further comprises a support, the membrane preferably has a porosity of 21 to 70%.

The porosity of the membrane may be determined by gas displacement pycnometry, e.g. using a pycnometer (especially the AccuPyc™ II 1340 gas displacement pycnometry system available from Micromeritics Instrument Corporation).

The porosity of the membrane is the amount of volume that can be accessed by external fluid or gas. This may be determined as described below. Preferably, the porosity of the membrane of the present invention is more than 20%.

When the membrane of the present invention includes a support, the thickness of the membrane including the support, in the dry state, is preferably 20 μm to 2,000 μm, more preferably 40 μm to 1,000 μm, and particularly preferably 70 μm to 800 μm.

When the membrane of the present invention does not comprise a support, the thickness of the membrane in a dry state is preferably 20 μm to 2,000 μm, more preferably 100 μm to 2,000 μm, and particularly preferably 150 μm to 2,000 μm.

When the membrane of the present invention includes a support, the thickness of the membrane including the support, when measured after storing for 12 hours in a 0.1 M NaCl solution, is preferably 10 μm to 4,000 μm, more preferably 20 μm to 2,000 μm and particularly preferably 20 μm to 1,500 μm.

When the membrane of the present invention does not comprise a support, the thickness of the membrane, when measured after storing for 12 hours in a 0.1 M NaCl solution, is preferably 10 μm to 4,000 μm, more preferably 50 μm to 4,000 μm and especially 70 μm to 4,000 μm.

If desired the composition may be applied to a support (especially a porous support) between steps (a) and (b) of the process according to the second aspect of the present invention. Step (b) may be performed on the composition which is present on and/or in the support. When the membrane is not required to comprise a support, the membrane may be peeled-off the support. Alternatively if the membrane is required to comprise a support then the membrane may be left on and/or in the support.

The composition may be applied to the support or the support may be immersed in the composition by various methods, for example, curtain coating, extrusion coating, air knife coating, slide coating, nip roll coating, forward roll coating, reverse roll coating, dip coating, kiss coating, rod bar coating, and spray coating. Coating of a plurality of layers can be performed simultaneously or sequentially. In simultaneous multilayer coating, curtain coating, slide coating, slot die coating, or extrusion coating is preferable.

The composition may be applied to a support at a temperature which assists the desired phase separation of the membrane from the composition. The temperature at which the composition is applied to the support (when present) is preferably below 80° C., more preferably between 10 and 60° C. and especially between 15 and 50° C.

When the membrane comprises a support, before the composition is applied to the surface of the support one may treat the surface of the support e.g. using a corona discharge treatment, a glow discharge treatment, a flame treatment, or an ultraviolet rays irradiation treatment. In this way one may improve the wettability and the adhesion of the support.

Step (b) optionally further comprises heating the composition.

Thus in a preferred process, the composition is applied continuously to a moving support, more preferably by means of a manufacturing unit comprising one or more composition application station(s), one or more irradiation source(s) for curing the composition, a membrane collecting station and a means for moving the support from the composition application station(s) to the irradiation source(s) and to the membrane collecting station.

The composition application station can be placed at the upstream position with respect to the irradiation source, and the irradiation source can be placed at the upstream position with respect to the composite membrane collecting station.

Preferably the curing of the composition is initiated within 60 seconds, more preferably within 15 seconds, particularly preferably within 5 seconds and most preferably within 3 seconds from when the composition is applied to the support or from when the support has been impregnated with the composition (when a support is used).

Light irradiation for photocuring is preferably performed for less than 10 seconds, more preferably for less than 5 seconds, particularly preferably for less than 3 seconds and most preferably for less than 2 seconds. In a continuous for preparing the membrane, the membrane may be irradiated continuously. The speed at which the composition is moved through the irradiation beam created by the irradiation source then determines the cure time and radiation dose.

Preferably the composition is cured by a process comprising irradiating the composition with ultraviolet (UV) light. The wavelength of the UV light used depends on the photoinitiator present in the composition and, for example, the UV light is UV-A (400 nm to 320 nm), UV-B (320 nm to 280 nm) and/or UV-C (280 nm to 200 nm).

When high intensity UV light is used to cure the composition a significant amount of heat may be generated. In order to prevent overheating, it is preferable to cool the lamp of the light source and/or the support/membrane with cooling air. When the composition is irradiated with a high dose of infrared light (IR light) together with a UV light, irradiation with UV light is preferably performed by using an IR reflecting quartz plate as a filter.

Examples of UV light sources include a mercury arc lamp, a carbon arc lamp, a low pressure mercury lamp, a medium pressure mercury lamp, a high pressure mercury lamp, a swirling flow plasma arc lamp, a metal halide lamp, a xenon lamp, a tungsten lamp, a halogen lamp, laser, and an ultraviolet ray emitting diode. A medium pressure or high pressure mercury vapor type ultraviolet ray emitting lamp is particularly preferable. Additionally, to modify the emission spectrum of a lamp, an additive such as metal halide may be present. A lamp having an emission maximum at a wavelength of 200 nm to 450 nm is particularly suitable.

The energy output of the radiation source is preferably 20 W/cm to 1000 W/cm and more preferably 40 W/cm to 500 W/cm, but if a desired exposure dose can be achieved, the energy output may be higher or lower than the aforementioned exposure dose. By the exposure intensity, curing of the film is adjusted. The exposure dose is measured in a wavelength range of UV-A by using a High Energy UV Radiometer (UV Power Puck (Registered Trademark) manufactured by EIT-Instrument Markets), and the exposure dose is preferably 40 mJ/cm² or greater, more preferably 100 mJ/cm² to 3,000 mJ/cm², and most preferably 150 mJ/cm² to 1,500 mJ/cm². The exposure time can be freely selected, and is preferably short, and most preferably less than 2 seconds.

The membrane of the present invention is particularly useful for purifying biomolecules and compositions comprising metal ions. The biomolecules include, for example, proteins, peptides, amino acids, anti-bodies and nucleic acids in biomedical applications.

The waterflux of the membrane of the present invention is preferably more than 100 l/(m²/bar/h), more preferably more than 200 l/(hr·m²·bar), especially more than 500 l/(m²/bar/hr) and especially preferably more than 1000 l/(m²/bar/hr).

In one embodiment the membrane has a water flux above of 200 to 500 l/(hr·m²·bar).

The water flux of the membranes according to the present invention may be determined as described below.

The swelling of the membranes of the present invention may be determined by measuring the volume of the membrane when dry and when wet with water (e.g. after being left in distilled water for 16 hours) and performing the following calculation:

$$Swelling = \frac{{Volume}_{wet} - {Volume}_{dry}}{{Volume}_{dry}} \times 100 \%$$

The swelling (i.e. % increase in volume) of the membranes in water is preferably less than 10%, more preferable less than 5%, especially less than 3.5% and most preferably 1.0% or less.

The membranes of the present invention are preferably affinity membranes. The membranes are particularly useful for affinity membrane filtration.

Preferably the membrane has a water permeability of at least 1.0 L/(h·m²·bar), more preferably at least 5.0 L/(h·m²·bar), especially at least 10 L/(h·m²·bar) and more especially at least 100 L/(h·m²·bar).

Preferably the membrane has the following properties (a), (b), optionally (c) and optionally (d):
(a) a water flux of at least 1 l/(hr·m²·bar);
(b) a porosity of 10% or more;
(c) a binding constant for a metal (e.g. copper) or a biomolecule of above 10⁴ M⁻¹; and
(d) when left in distilled water for 16 hours increases in volume by less than 3.5%.

According to a third aspect of the present invention there is provided the use of a membrane according to the first aspect of the present invention for detecting, filtering and/or purifying biomolecules and/or compositions comprising metal ions.

The membranes according to the first aspect of the present invention may be used for purifying compositions comprising metal-ions (e.g. one or more metal-ions and optionally contaminants) from metal ions by, for example, contacting the membranes with a fed solution comprising one or more species of metal-ions, allowing the ligand present in the membrane to bind to one or more of the metal ions present in the composition and then collecting the remainder of the feed solution which has a lower content of the metal ions due to the metal ions being bound to the ligand present in the membrane. The metal-ions from the feed solution are attracted to the ligand group from component (i). The affinity of the ligand groups for the metal-ions depends on the binding capacity or stability constant of the ligand group for the particular metal-ions concerned.

The membranes of the present invention may be used to purify feed solutions by a number of processes, including use of the membranes in AMF, in size-exclusion chromatography (e.g. where the pores of the membrane are used to remove metal-ions or colloids containing metals or agglomerates of metal particles based on their size (i.e., physical exclusion)) and in affinity chromatography (e.g. where liquids are purified according to the binding capacity (stability constant) of the metal-ions with the ligand groups in the membrane (i.e. covalent or non-covalent interactions)).

Preferably the membrane has a metal removal capacity for Cu of 150 to 1000 μmol/g of the membrane, more preferably of 197 to 661 μmol/g of the membrane.

According to a fourth aspect of the present invention there is provided use of a membrane according to the first aspect of the present invention for detecting, filtering and/or purifying biomolecules.

The membranes according to the first aspect of the present invention may be used for filtering, and/or purifying biomolecules by eluting solutions containing biomolecules, especially biomolecules which carry a molecular unit which has an affinity for the ligand group. The biomolecules are attracted to and bind to the ligand group of the membrane derived from component (i).

The membranes of the present invention may be used to purify biomolecules by a number of processes, including use of the membranes in size-exclusion chromatography (e.g. where the pores of the membrane are used to separate or purify biomolecules based on their size (i.e., physical exclusion)) and in affinity chromatography (e.g. where biomolecules are purified or separated according to the binding capacity (stability constant) of the biomolecules with the ligand groups in the membrane (i.e. covalent or non-covalent interactions)).

The membranes according to the first aspect of the present invention may be used for filtering, and/or purifying biomolecules by analogous techniques to those described above for biomolecules, especially by affinity membrane filtration.

The membranes according to the first aspect of the present invention may be used for detecting biomolecules by techniques involving the detection of colour, especially when the biomolecules comprise a fluorescent or coloured marker.

Thus a further aspect of the present invention comprises a process for purifying an impure composition comprising a desired biomolecule comprising the steps:
(i) contacting the composition with a membrane according to the present invention and allowing the desired biomolecule to bond to the ligand group:
(ii) washing membrane product of step (i) to remove impurities from the composition such that the desired biomolecule remains bound to the ligand group; and
(iii) removing the desired biomolecule from the ligand and collecting the desired biomolecule.

Typically step (ii) comprises washing the membrane product of step (i) with an aqueous buffer which ensures that most or all of the desired biomolecule remains bound to the ligand group and impurities (e.g. biomolecules which are not desired) are washed away. Step (iii) may be performed by washing the membrane with an aqueous solution comprising a buffer having a different pH to the buffer used in step (i) or an aqueous solution comprising a chemical which has a higher affinity for the ligand than the desired biomolecule.

In a still further aspect of the present invention there is provided a process for purifying a feed composition comprising metal ions comprising the steps of contacting the feed composition with a membrane according to the present invention and allowing the metal ions to bond to the ligand group such that a composition is obtained which has a lower metal content than the feed composition.

Preferably the process for purifying a biomolecule and/or separating a biomolecule from other biomolecules comprises membrane size-exclusion chromatography or affinity chromatography.

The membranes may of course be used for other purposes too.

The invention will now be illustrated by the following, non-limiting examples in which all parts and percentages are by weight unless otherwise specified. The following abbreviations are used in the Examples:

| | |
|---|---|
| FO-2223-10 | is a non-woven, polypropylene-based, porous cloth of thickness |
| | 100 μm obtained from Freudenberg Group. This acts as a porous |
| | support. |
| IPA | is isopropanol. |
| AMPS-Na | is the sodium salt of 2-acrylamido-2-methylpropane sulphonic acid |
| | having the structure shown below (from Sigma Aldrich). |
| CN132 | is a cross-linking agent having no ligand groups and having the |
| | structure shown below (from Sartomer). |
| MBA | is a cross-linking agent having no ligand groups and having the |
| | structure shown below (from Sigma-Aldrich). |
| (M49) | is a cross-linking agent ha ving a ligand group and anionic groups and |
| | having the structure shown (from FUJIFILM). (M49) may be prepared |
| | by the method described for compound (M-11) in EP2,965,803, |
| | paragraph [0211]. |
| BAMPS | Is 1,1-bis(acryloylamido)-2-methylpropane-2-sulphonic acid (a cross- |
| M50 | linking agent comprising a ligand group). |
| | is a monofunctional reactive monomer with di-(2-picoyl)amine ligand |
| | functionality. M50 may be prepared by the method described for |
| | compound (11) in Sterk, M. and Bannwarth, W. (2015), Modulation of |
| | Reactivities of Dienophiles for Diels-Alder Reactions via |
| | Complexation of α, β-Unsaturated Chelating Amides. HCA, 98: 287- |
| | 307. |
| (M3) | may be prepared by the method described below in the Examples. |
| (M7) | may be prepared by the method described below in the Examples. |
| (M19) | may be prepared by the method described below in the Examples. |
| V-50 | is a thermal initiator having the structure as depicted below and can |
| | be obtained from FUJIFILM Wako Pure Chemical Corporation. |

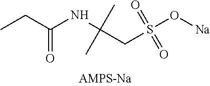

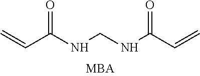

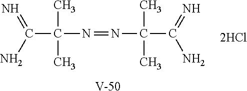

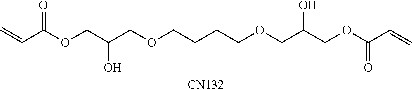 [SMILES: "CN132" ; C=CC(=O)OCC(O)COCCCCOCC(O)COC(=O)C=C]

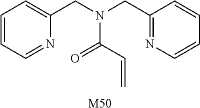

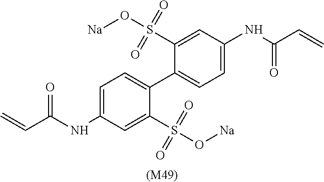

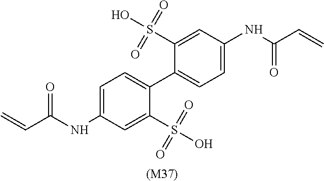

The water flux, metal removal capacity, porosity and thickness of the membranes described in the Examples and Comparative Example were measured as described below:

### I) Water Flux (L/(m²/Bar/Hr)) of the Membrane

Water flux of the membranes was measured using a device where the weight of water passing through the membrane was measured over time. A column of feed solution (pure water) was brought into contact with the membrane under evaluation and the feed solution was forced through the membrane by a constant applied air pressure on top of the water column. By achieving a constant flow of water at a constant applied pressure, the water flux could be determined.

Typically the membrane under evaluation was stored for 12 hours in pure water prior to use. The feed solution (250 ml of pure water) was brought into contact with the membrane (film contact area of 12.19 cm²). The water column was closed and pressurized with air pressure and the membrane was flushed with one water column (250 ml). The feed solution was refreshed and a constant air pressure of 100 mbar was applied. Finally, the measurements were performed by monitoring the weight by balance at a constant flow.

II) Metal Removal Capacity (μmol/q) of the Membrane

Prior to measuring a membrane's metal removal capacity, the membrane was weighed in the dry state. The membrane was then flushed 3× with demineralized water and 3× with iso-propanol. Subsequently, the membrane was placed in a filter holder and 50 ml of a 2.0 mM solution of CuSO₄ in MeOH was passed through the membrane. The membrane was digested in acid solution and analysed by ICP-OES to determine the amount of Cu per unit weight of membrane in μmol/g (i.e. micromoles of copper per gram of dry membrane).

### III) Mean Flow Pore Size (MFP) of the Membrane

The mean flow pore size of the membrane was measured using a porometer, e.g. a Porolux™ porometer. The membrane to be tested was fully wetted with a wetting fluid (e.g. Porefil™ wetting Fluid, an inert, non-toxic, fluorocarbon wetting fluid with zero contact angle). Place the wetted membrane in the sample holder of the porometer and apply a pressure of up to 35 mbar nitrogen gas. The porometer can then measure the flow of gas through the sample, as the liquid is displaced out of the porous membrane. This will provide the bubble point, maximum pore size, mean flow pore size, minimum pore size, average pore size distribution (of uniform materials) and air permeability of the membrane under test. The mean flow pore diameter is the pore size at which 50% of the total gas flow can be accounted. This means that half the flow is through pores larger than this diameter.

### IV) Porosity (%) of the Membrane

The porosity of the membrane under evaluation was determined from the apparent density (ρₐₚₚₐᵣₑₙₜ₎ and the real density of the membrane. The ρₐₚₚₐᵣₑₙₜ was measured in air by weighing the membrane and determining its volume from the dimensions of the membrane (length, width and thickness). The real density of the membrane was determined from pycnometer measurements of the membrane with known weight under helium atmosphere. The Helium occupied the pores of the membrane with known weight, and therefore the volume of polymer could be determined. From this the porosity could be determined according to Formula (1):

$$Porosity = \frac{{ρ}_{real} - {ρ}_{apparent}}{{ρ}_{real}} \times 100 \% \qquad ( 1 )$$

The pycnometer used was the AccuPyc™ II 1340 gas displacement pycnometry system from Micromeritics Instrument Corporation.

### V) Thickness (μm) of the Membrane

The thickness of the membranes was determined by contact mode measurement. The measurements were performed at five different positions of the membrane and the average thickness of these five measurements in μm was calculated.

### VI) Swelling (%)

The swelling (%) of the membranes in water was determined as follows: A circular membrane of 47 mm diameter was dried by open air for 16 hours to give a dry membrane. The volume of the dry membrane was then determined by measuring the width and thickness in 2-3 locations. The average of the dimensions are taken and the volume is calculated according to the following formula:

$$Volume = {π ( \frac{width}{2} )}^{2} * thickness$$

The dry membrane was then allowed to stand in distilled water (100 cm³) for 16 hours to give a wet membrane. The volume of the wet membrane was then determined by measuring the width and the thickness again in 2-3 locations. Also the average of the dimensions are taken and the volume is calculated according to the formula above. The swelling % was then determined by performing the following calculation:

$$Swelling = \frac{{Volume}_{wet} - {Volume}_{dry}}{{Volume}_{dry}} \times 100 \%$$

VII) Pore Surface Area, S_{BET} (M²/g):

The method for measuring the pore surface area was in accordance with ISO 9277.

Samples of each membrane under test were degassed in vacuum at 25° C. for 16 hours. The dry weight of the resultant, degassed membrane was recorded.

The ability of the degassed samples to absorb nitrogen gas was then recorded at 77 K using a Micromeritics TriStar II 3020 adsorption analyser as follows.

A cell containing a degassed sample of membrane under test was evacuated and then cooled using liquid nitrogen to a temperature of 77 Kelvin. Portions of nitrogen gas were then dosed into the cell and the nitrogen gas was partly adsorbed onto the surface of the membrane under test until an equilibrium was reached with the gas phase. In this way adsorption and desorption points were recorded at different pressures and an adsorption and desorption isotherm was constructed. Adsorbed nitrogen first formed a quasi-monolayer on the membrane surface whereas further increases in pressure resulted in the formation of multilayers. In the region where monolayer and multilayers were formed, the specific surface area (S_{BET}) was determined according to the BET (Brunauer, Emmet and Teller) theory. This model is applicable to non-porous and meso- and macroporous materials and adsorption points in the relative pressure range between 0.05 and 0.25 were used.

### Preparation of Cross-Linking Agents Comprising at Least One Ligand Group

### Preparation of (M3)

The di(2-picolyl)amine (50 mg, 0.25 mmol) was dissolved in water (0.85 ml) and cooled to 0° C. Subsequently, N, N′-{[(2-acrylamido-2-[(3-acrylamidopropoxy)methyl]propane-1,3-diyl)bis(oxy)]bis(propane-1,3-diyl)}diacrylamide (531 mg, 0.75 mmol) in a mixture of iso-propanol (0.25 ml) and water (0.8 ml) was added at 0° C. and slowly warmed to 80° C. while stirring. The reaction was monitored by TLC and completed after 75 hours. The solvent was removed and the crude material was purified by silica gel column chromatography (Ethyl acetate 4:1 Methanol) to give M3 as a colourless oil (177 mg) in quantitative yield.

The compound M3 was characterised by ¹H NMR (62 MHz, CDCl₃, δ): 8.56 (m, 2H), 7.78-6.91 (m, 6H), 6.36-6.16 (m, 6H), 5.66-5.46 (m, 3H), 4.69 (s, 1H), 3.84-3.10 (m, 22H), 2.88 (m, 2H), 2.50 (m, 5H) and 1.86-1.66 (m, 6H).

### Preparation of (M7)

N,N′,N″,N′″-tetraacryloyltriethylenetetramine (91 mg, 0.25 mmol) was dissolved in water (1 ml) and di(2-picolyl)amine (100 mg, 0.50 mmol) was added. Subsequently, 4-methoxyphenol (1.24 mg, 0.01 mmol) and 1,8-diazabicyclo[5.4.0]undec-7-ene (19 mg, 0.125 mmol) were also added and stirred at room temperature. The reaction was monitored by reversed phase TLC and complete after 5 days. The solvent was removed and the crude compound consisted of a statistical distribution of products with M7 as the main component according to LC-MS analysis. M7 was directly used without further purification.

The compound M7 was characterised by ¹H NMR (62 MHz, CDCl₃, δ): 8.60-8.52 (m, 4H), 7.97-7.38 (m, 12H), 6.07-5.63 (m, 6H), 5.66-5.46 (m, 3H), 4.69 (br. s, 2H), 3.84 (s, 8H), 3.53-3.01 (m, 16H), and 2.50-2.30 (m, 4H).

### Preparation of (M19)

N-Boc-1,2-ethylenediamine (9.61 g, 60 mmol) was dissolved in EtOAc (200 mL), and a solution of anhydrous potassium carbonate (124.4 g, 900 mmol) in water (200 mL) was added. The resulting mixture was vigorously stirred at 0° C. and acryloyl chloride (18.2 mL, 223.7 mmol) was added dropwise. Next, the resulting solution was allowed to reach room temperature and stirred overnight. The organic layer was separated from the aqueous layer, after which the aqueous layer was extracted three times with EtOAc (3×200 ml). The organic layers were combined and dried with Na₂SO₄, filtered, and evaporated to give N-Boc-1,2-ethylenediamine acrylamide as a white solid (12.6 g, 98%). The N-Boc-1,2-ethylenediamine acrylamide (12.6 g, 58.9 mmol) was dissolved in DCM (50 mL), and trifluoroacetic acid (50 mL) was slowly added. The resulting mixture was stirred at room temperature. After 30 minutes, deprotection was complete (as confirmed by TLC). Afterwards, DCM and TFA were evaporated to give N-(2-aminoethyl)acrylamide as a TFA salt (13.4 g) in quantitative yield. This was directly used by redissolving in anhydrous DMF (45 ml) and addition of the diethylenetriaminepentaacetic dianhydride (2.5 g, 7.0 mmol). Next, triethylamine was added dropwise until the pH of the solution remained stable at pH 8-9 and the resulting mixture was stirred overnight at room temperature. The reaction was monitored by TLC and afterwards the solvents were concentrated and the crude material was purified by reverse phase C18 silica gel column chromatography with water as eluent to give M19 as a colourless oil (1.97 g, 48%).

The compound M19 was characterised by ¹H NMR (62 MHz, DMSO-d6, δ): 8.58 (s, 2H), 8.16 (s, 2H), 6.32 (m, 2H), 6.08 (d, 2H, J=32 Hz), 5.56 (d, 2H, J=32 Hz), 3.28 (m, 26H).

### EXAMPLES 1 TO 39

### (ai) Preparation of Compositions

Compositions 1 to 39 were prepared by mixing the ingredients indicated in Table 1 below in the specified amounts. In Table 1, component (i) had the structure identified above in the description, component (ii) was as described in Table 1, component (iii) was Irgacure™ 1173 (a photoinitiator), and component (iv) when present was M50 as described above. The compositions were each applied to a porous support (FO-2223-10) by the process described in more detail further in this specification.

TABLE 1
Compositions

| | | | Component | | | | |
|---|---|---|---|---|---|---|---|
| Composition | | Amount of | (ii) | | | | |
| and | Component | Component | water | IPA | Component | Component | Value of |
| Example | (i) | (i) (wt %) | (wt %) | (wt %) | (iii) (wt %) | (iv) (wt %) | Z |
| 1 | (M3) | 28.97 | 27.03 | 43.81 | 0.19 | 0.00 | 0.993 |
| 2 | (M3) | 26.89 | 32.27 | 40.66 | 0.18 | 0.00 | 0.993 |
| 3 | (M3) | 25.09 | 36.80 | 37.94 | 0.17 | 0.00 | 0.993 |
| 4 | (M3) | 23.52 | 40.76 | 35.56 | 0.16 | 0.00 | 0.993 |
| 5 | (M3) | 22.13 | 44.26 | 33.46 | 0.15 | 0.00 | 0.993 |
| 6 | (M3) | 27.31 | 25.49 | 47.02 | 0.18 | 0.00 | 0.993 |
| 7 | (M3) | 25.83 | 24.11 | 49.89 | 0.17 | 0.00 | 0.993 |
| 8 | (M3) | 24.51 | 22.87 | 52.46 | 0.16 | 0.00 | 0.994 |
| 9 | (M3) | 23.31 | 21.76 | 54.77 | 0.16 | 0.00 | 0.993 |
| 10 | (M3) | 30.31 | 26.52 | 42.98 | 0.19 | 0.00 | 0.994 |
| 11 | (M3) | 33.00 | 23.00 | 43.81 | 0.19 | 0.00 | 0.994 |
| 12 | (M3) | 40.00 | 26.81 | 33.00 | 0.19 | 0.00 | 0.995 |
| 13 | (M7) | 28.97 | 27.03 | 43.81 | 0.19 | 0.00 | 0.993 |
| 14 | (M7) | 26.89 | 32.27 | 40.66 | 0.18 | 0.00 | 0.993 |
| 15 | (M7) | 25.09 | 36.80 | 37.94 | 0.17 | 0.00 | 0.993 |
| 16 | (M7) | 23.52 | 40.76 | 35.56 | 0.16 | 0.00 | 0.993 |
| 17 | (M7) | 22.13 | 44.26 | 33.46 | 0.15 | 0.00 | 0.993 |
| 18 | (M7) | 27.31 | 25.49 | 47.02 | 0.18 | 0.00 | 0.993 |
| 19 | (M7) | 25.83 | 24.11 | 49.89 | 0.17 | 0.00 | 0.993 |
| 20 | (M7) | 24.51 | 22.87 | 52.46 | 0.16 | 0.00 | 0.994 |
| 21 | (M7) | 23.31 | 21.76 | 54.77 | 0.16 | 0.00 | 0.993 |
| 22 | (M7) | 30.31 | 26.52 | 42.98 | 0.19 | 0.00 | 0.994 |
| 23 | (M7) | 33.00 | 23.00 | 43.81 | 0.19 | 0.00 | 0.994 |
| 24 | (M7) | 40.00 | 26.81 | 33.00 | 0.19 | 0.00 | 0.995 |
| 25 | (M19) | 28.97 | 27.03 | 43.81 | 0.19 | 0.00 | 0.993 |
| 26 | (M19) | 26.89 | 32.27 | 40.66 | 0.18 | 0.00 | 0.993 |
| 27 | (M19) | 25.09 | 36.80 | 37.94 | 0.17 | 0.00 | 0.993 |
| 28 | (M19) | 23.52 | 40.76 | 35.56 | 0.16 | 0.00 | 0.993 |
| 29 | (M19) | 22.13 | 44.26 | 33.46 | 0.15 | 0.00 | 0.993 |
| 30 | (M19) | 27.31 | 25.49 | 47.02 | 0.18 | 0.00 | 0.993 |
| 31 | (M19) | 25.83 | 24.11 | 49.89 | 0.17 | 0.00 | 0.993 |
| 32 | (M19) | 24.51 | 22.87 | 52.46 | 0.16 | 0.00 | 0.994 |
| 33 | (M19) | 23.31 | 21.76 | 54.77 | 0.16 | 0.00 | 0.993 |
| 34 | (M19) | 30.31 | 26.52 | 42.98 | 0.19 | 0.00 | 0.994 |
| 35 | (M19) | 33.00 | 23.00 | 43.81 | 0.19 | 0.00 | 0.994 |
| 36 | (M19) | 40.00 | 26.81 | 33.00 | 0.19 | 0.00 | 0.995 |
| 37 | (M3) | 40.00 | 15.81 | 24.00 | 0.19 | 20.00 | 0.665 |
| 38 | (M3) | 40.00 | 17.81 | 27.00 | 0.19 | 15.00 | 0.725 |
| 39 | (M3) | 40.00 | 20.31 | 31.00 | 0.19 | 8.50 | 0.822 |

(aii) Application of the Compositions to a Support

The compositions described in Table 1 above were each independently applied to a porous support (FO-2223-10) at 20° C. using a tabletop coating machine (manufactured by TQC, Model AB3000 Automatic film applicator). The supports were attached to a glass plate and the compositions were applied to the supports at a speed of about 1 cm/sec using a wire bar (a stainless steel bar on which a wire of 150 μm had been wound at 1 lap/3 cm (length direction). Any excess composition and air bubbles were removed from the coated supports using a 12 μm wire bar.

### (b) Curing the Compositions to Form the Membrane

The compositions present on the supports were cured by irradiation with UV using a Light Hammer LH6 UV exposure machine (manufactured by Fusion UV Systems, Inc.). The Light Hammer machine was fitted with a Model H-bulb (100% strength). The coated supports were passed through the Light Hammer machine at a speed of 10m/min to expose the composition to the UV light from the H-bulb. The curing time was 0.8 seconds. The exposure time was 0.71 seconds. The resultant membranes were removed from the glass plate and was stored in a polyolefin bag.

### EXAMPLE 40

In this Example a membrane was prepared which did not comprise a support and the curing was thermal curing. A composition was prepared exactly as described for Example 1 except that in place of Irgacure™ 1173 there was used was V-50 (a thermal initiator, 2 wt %) having the structure shown above. Z had a value of 0.935. The composition (5.0 cm³) was placed in a glass vial having a capacity of 25 cm³. The vial was sealed and placed in a vacuum oven at 50° C. for 60 minutes. The oven was cooled down to room temperature and the vial was removed from the oven. The resultant membrane was then removed from the vial.

### EXAMPLE 41

Example 1 was repeated except that the membrane did not comprise a porous support.

The composition described in Example 1 was applied to the glass plate at 20° C. using a tabletop coating machine (manufactured by TQC, Model AB3000 Automatic film applicator) at a speed of about 1 cm/sec using a wire bar (a stainless steel bar on which a wire of 150 μm had been wound at 1 lap/3 cm (length direction).

The composition present on the glass plate was cured by irradiation with UV using a Light Hammer LH6 UV exposure machine (manufactured by Fusion UV Systems, Inc.). The Light Hammer machine was fitted with a Model H-bulb (100% strength). The coated sample was passed through the Light Hammer machine at a speed of 10m/min to expose the composition to the UV light from the H-bulb. The curing time was 0.8 seconds. The exposure time was 0.71 seconds. The resultant membrane was removed from the glass plate and was stored in a polyolefin bag.

### EXAMPLE 42C

### (a) Preparation and Application of a Composition

A composition was prepared by mixing the following components in the specified amounts: (M49) (31.68 wt %); AMPS-Na (6.49 wt %); ethanolamine (0.38 wt %); iso-propanol (30.19 wt %); water (30.49 wt %); and Irgacure™ 1173 (0.77 wt %). Z had a value of 0.814. The composition was applied to a non-woven support (FO-2223-10) at 20° C. using a tabletop coating machine (manufactured by TQC, Model AB3000 Automatic film applicator). The support was attached to a glass plate and the composition was applied to the support at a speed of about 1 cm/sec using a wire bar (a stainless steel bar on which a wire of 150 μm had been wound at 1 lap/3 cm (length direction). Any excess composition and air bubbles were removed from the coated support using a 12 μm wire bar.

### (b) Curing the Compositions to Form the Membrane

The composition present on the support was cured by irradiation with UV using a Light Hammer LH6 UV exposure machine (manufactured by Fusion UV Systems, Inc.). The Light Hammer machine was fitted with a Model H-bulb (100% strength). The coated support was passed through the Light Hammer machine at a speed of 10m/min to expose the composition to the UV light from the H-bulb and subsequently the D-bulb. The curing time was 0.8 seconds. The exposure time was 1.42 seconds. The resultant membrane was removed from the glass plate and was stored in a polyolefin bag.

### (c) Acid Treatment of the Membrane

The membrane arising from Step (b) was washed three times with 0.2 M H₂SO₄ (aq.), which resulted in a membrane akin to polymerised monomer (M37)(i.e. non-ionic and in free acid instead of salt form). The resultant membrane had a higher binding affinity for metal ions than when in the salt form. The resultant membrane had a dry thickness of 110 μm, an metal removal capacity of 353 μmol/g, a water flux of 305 l/m²/bar/hr, an average pore size of 250 nm and a swelling in water of 2.3%.

### Properties of the Resultant Membranes

The membranes obtained in Examples 1 to 41 and 42c had the properties described in Table 2 below:

TABLE 2
Membrane Properties

| | | Metal | | | | Pore |
|---|---|---|---|---|---|---|
| | | removal | | | | surface |
| | Waterflux | capacity | Porosity | Swelling | MFP | area S_{BET} |
| Example | (l/(m²/bar/hr)) | (μmol/g) | (%) | (%) | (nm) | (m²/g) |
| 1 | 222 | 257 | 41.0 | 1.0 | 245 | 912 |
| 2 | 233 | 239 | 43.1 | 0.8 | 251 | 958 |
| 3 | 243 | 223 | 44.9 | 0.8 | 253 | 998 |
| 4 | 251 | 209 | 46.5 | 0.9 | 248 | 1033 |
| 5 | 259 | 197 | 47.9 | 0.6 | 246 | 1064 |
| 6 | 231 | 243 | 42.7 | 0.8 | 250 | 949 |
| 7 | 239 | 230 | 44.2 | 0.5 | 252 | 982 |
| 8 | 246 | 218 | 45.5 | 0.7 | 253 | 1011 |
| 9 | 252 | 207 | 46.7 | 0.8 | 248 | 1038 |
| 10 | 215 | 269 | 39.7 | 0.9 | 248 | 882 |
| 11 | 210 | 293 | 37.0 | 0.7 | 253 | 822 |
| 12 | 202 | 355 | 30.0 | 0.9 | 251 | 667 |
| 13 | 222 | 479 | 41.0 | 1.0 | 200 | 910 |
| 14 | 233 | 445 | 43.1 | 0.7 | 198 | 960 |
| 15 | 243 | 415 | 44.9 | 0.9 | 195 | 1000 |
| 16 | 251 | 389 | 46.5 | 1.0 | 196 | 1031 |
| 17 | 259 | 366 | 47.9 | 0.6 | 202 | 1062 |
| 18 | 231 | 451 | 42.7 | 1.0 | 202 | 947 |
| 19 | 239 | 427 | 44.2 | 0.8 | 199 | 980 |
| 20 | 246 | 405 | 45.5 | 0.9 | 198 | 1009 |
| 21 | 252 | 385 | 46.7 | 0.8 | 199 | 1036 |
| 22 | 215 | 501 | 39.7 | 0.7 | 195 | 880 |
| 23 | 210 | 546 | 37.0 | 0.7 | 201 | 821 |
| 24 | 202 | 661 | 30.0 | 0.5 | 200 | 665 |
| 25 | 222 | 402 | 41.0 | 0.6 | 150 | 921 |
| 26 | 233 | 373 | 43.1 | 0.8 | 146 | 968 |
| 27 | 243 | 348 | 44.9 | 0.8 | 146 | 1008 |
| 28 | 251 | 326 | 46.5 | 0.8 | 151 | 1043 |
| 29 | 259 | 307 | 47.9 | 0.6 | 154 | 1075 |
| 30 | 231 | 379 | 42.7 | 0.7 | 147 | 958 |
| 31 | 239 | 358 | 44.2 | 0.6 | 149 | 991 |
| 32 | 246 | 340 | 45.5 | 0.5 | 150 | 1021 |
| 33 | 252 | 323 | 46.7 | 0.6 | 150 | 1048 |
| 34 | 215 | 420 | 39.7 | 0.9 | 145 | 891 |
| 35 | 210 | 458 | 37.0 | 0.7 | 146 | 831 |
| 36 | 202 | 555 | 30.0 | 0.7 | 153 | 673 |
| 37 | 407 | 325 | 25.5 | 1.3 | 220 | 753 |
| 38 | 450 | 380 | 27.8 | 1.2 | 217 | 788 |
| 39 | 443 | 376 | 24.9 | 1.1 | 244 | 726 |
| 40 | 308 | 375 | 35.9 | 3.5 | 155 | 805 |
| 41 | 410 | 312 | 38.6 | 1.8 | 502 | 867 |
| 42c | 305 | 353 | 41.0 | 2.3 | 244 | 920 |

### COMPARATIVE EXAMPLES 1 TO 8

### Preparation of Compositions

Comparative composition CEx1 to CEx7 were prepared by mixing the ingredients indicated in Table 4 below in the specified amounts. In Table 4, component (i) had the structure identified above in the description, component (ii) consisted of the components specified in Table 4, component (iii) was Irgacure™ 1173 in the amounts indicated.

The membranes obtained in Comparative Examples CEx1 to CEx7 were prepared using the method described for Example 1 above, except that the compositions indicated in Table 4 below were used. The support used in all cases was FO-2223-10.

CEx8 was prepared according to the following procedure:

### (a) Preparation and Application of a Composition

A composition was prepared by mixing the following components in the specified amounts: (M49) (31.68 wt %); AMPS-Na (6.49 wt %); ethanolamine (0.38 wt %); iso-propanol (30.19 wt %); water (30.49 wt %); and Irgacure™ 1173 (0.77 wt %). Z had a value of 0.814. The composition was applied to a non-woven support (FO-2223-10) at 20° C. using a tabletop coating machine (manufactured by TQC, Model AB3000 Automatic film applicator). The support was attached to a glass plate and the composition was applied to the support at a speed of about 1 cm/sec using a wire bar (a stainless steel bar on which a wire of 150 μm had been wound at 1 lap/3 cm (length direction). Any excess composition and air bubbles were removed from the coated support using a 12 μm wire bar.

### (b) Curing the Compositions to Form the Membrane

The composition present on the support was cured by irradiation with UV using a Light Hammer LH6 UV exposure machine (manufactured by Fusion UV Systems, Inc.). The Light Hammer machine was fitted with a Model H-bulb (100% strength). The coated support was passed through the Light Hammer machine at a speed of 10m/min to expose the composition to the UV light from the H-bulb and subsequently the D-bulb. The curing time was 0.8 seconds. The exposure time was 1.42 seconds. The resultant membrane was removed from the glass plate and was stored in a polyolefin bag.

The resultant membrane had a low affinity for metals (lower than the membrane from Example 42-c).

TABLE 4
Compositions Used in Comparative Examples CEx1 to CEx7

| | Cross-linking | | | | | | | | |
|---|---|---|---|---|---|---|---|---|---|
| Comparative | agent | | Component (ii) | | | | Component (iv) | | |
| Example | | Amount | Water | IPA | Other | Component | | Amount | Value |
| # | Name | (wt %) | (wt %) | (wt %) | solvents | (iii) (wt %) | Name | (wt %) | of Z |
| CEx1 | CN132 | 37.50 | 49.00 | 12.50 | 0.00 | 1.00 | None | 0.00 | 0.000 |
| CEx2 | MBA | 1.30 | 14.36 | 0.00 | 9.57% | 0.11 | None | 0.00 | 0.000 |
| | | | | | DMF, | | | | |
| | | | | | 61.77% | | | | |
| | | | | | Dioxane, | | | | |
| | | | | | 1.69% | | | | |
| | | | | | Glycerol | | | | |
| CEx3 | None | 0.00 | 27.03 | 43.81 | 0.00 | 0.19 | M50 | 28.97 | 0.000 |
| CEx4 | BAMPS | 37.49 | 37.29 | 0.00 | 0.00 | 0.50 | AMPS- | 24.72 | 0.598 |
| | (Component | | | | | | Na | | |
| | (i)) | | | | | | | | |
| CEx5 | M3 | 5.90 | 23.38 | 20.53 | 0.00 | 0.19 | M50 | 50.00 | 0.105 |
| CEx6 | M3 | 5.00 | 35.81 | 44.00 | 0.00 | 0.19 | M50 | 15.00 | 0.247 |
| CEx7 | M3 | 5.90 | 49.00 | 38.91 | 0.00 | 0.19 | M50 | 6.00 | 0.488 |

### Properties of the Resultant Comparative Membranes

The membranes obtained in Comparative Examples CEx1 to CEx8 had the properties described in Table 5 below:

TABLE 5
Properties of the Comparative Membranes

| | | Metal | | | | Pore |
|---|---|---|---|---|---|---|
| | | removal | | | | surface |
| | Waterflux | capacity | Porosity | Swelling | MFP | area S_{BET} |
| Example | (l/(m²/bar/hr)) | (μmol/g) | (%) | (%) | (nm) | (m²/g) |
| CEx1 | 4228 | 0 | 33.3 | 3.3 | 448 | 734 |
| CEx2 | 10147 | 0 | 57.1 | 80.1 | 1012 | 110 |
| CEx3 | 1125 | 52 | 41.2 | 80.0 | 0 | 185 |
| CEx4 | 0 | 0 | 5.0 | 11.2 | 0 | 0 |
| CEx5 | 120422 | 56 | 62.2 | 33.8 | 0 | 0 |
| CEx6 | 122008 | 45 | 60.0 | 45.8 | 0 | 0 |
| CEx7 | 122407 | 32 | 58.9 | 27.4 | 0 | 0 |
| CEx8 | 310 | 101 | 32.0 | 2.4 | 249 | 711 |

1-29. (canceled)

30. A porous membrane obtained by a process comprising curing a composition comprising:
(i) cross-linking agent(s) comprising at least one ligand group L which comprises a compound of the Formula (1) or Formula (2):
(L)q-(A)m-(R)n Formula (1)
(R)n-1-(A)m-(L)q-(A)m-(R)n-1 Formula (2)
wherein:
each L independently is a ligand group capable of binding Cu;
each A independently is an organic linking group;
each R independently is polymerisable group;
q has a value of at least 1;
each m independently has a value of 0 or 1; and
each n independently has a value of at least 2;
(ii) inert solvent(s);
(iii) polymerization initiator(s); and
(i) optionally monomer(s) other than component (i) which are reactive with component (i);
wherein the composition satisfies the following equation:
Z=wt(i)/(wt(i)+wt(iii)+wt(iv))
wherein:
Z has a value of at least 0.6;
wt(i) is the number of grams of component (i) present in the composition;
wt(iii) is the number of grams of component (iii) present in the composition; and
wt(iv) is the number of grams of component (iv) present in the composition;
and wherein said membrane has a Cu removal capacity between 150 and 1000 μmol/g of the membrane determined by inductively coupled plasma-optical emission spectrometry.

31. The membrane according to claim 30 wherein the composition comprises 1.0 to 80.0 wt % of cross-linking agent(s) (i), 98.9 to 19.9 wt % inert solvent(s) (ii) and 0.1 to 5.0 wt % polymerization initiator(s)(iii).

32. The membrane according to claim 30 having a mean flow pore size of 100 nm to 5,000 nm as determined by a porometer with the membrane under an applied pressure of up to 35 mbar nitrogen gas and/or a porosity of 10% or more as determined by pycnometer measurements of the membrane under helium atmosphere according to formula
Porosity=pᵣₑₐₗ−pₐₚₚₐᵣₑₙₜ/pᵣₑₐₗ×100%.

33. The membrane according to claim 30 wherein the membrane is free from cationic and anionic groups.

34. The membrane according to claim 30 wherein component (i) is completely dissolved in component (ii) and the membrane is insoluble in component (ii).

35. The membrane according to claim 34 having a mean flow pore size of 100 nm to 5,000 nm as determined by a porometer with the membrane under an applied pressure of up to 35 mbar nitrogen gas and/or a porosity of 10% or more as determined by pycnometer measurements of the membrane under helium atmosphere according to formula 1
Porosity=pᵣₑₐₗ−pₐₚₚₐᵣₑₙₜ/pᵣₑₐₗ×100%.

36. The membrane according to claim 34 wherein component (ii) comprises isopropanol and water.

37. The membrane according to claim 30 wherein the curing comprises photocuring.

38. The membrane according to claim 30 wherein the curing comprises polymerisation-induced phase separation of the membrane from the composition.

39. The membrane according to claim 30 which further comprises a porous support.

40. The membrane according to claim 30 which has the following properties (a), (b), optionally (c) and optionally (d):
(a) a water flux of at least 1 l/(hr·m²·bar);
(b) a porosity of 10% or more;
(c) a binding constant for copper of above 10⁴ M⁻¹; and
(d) when left in distilled water for 16 hours increases in volume by less than 3.5%.

41. Use of a membrane according to claim 34 for detecting, filtering and/or purifying biomolecules.

42. Use of a membrane according to claim 34 for detecting metal ions or for filtering and/or purifying compositions comprising metal-ions.

43. A process for purifying a biomolecule and/or separating a biomolecule from other biomolecules comprising contacting the biomolecules with a membrane according to claim 30.

44. A process for purifying metal-ions and/or separating metal-ions from other particles or ionic species comprising contacting the metal-ions with a membrane according to claim 30.

45. The process according to claim 44 wherein the process comprises membrane size-exclusion chromatography or ion exchange chromatography.
